(19) United States
(12) Patent Application Publication
(10) Pub. No.: US 2006/0009465 A1
(43) Pub. Date: Jan. 12, 2006

(54) Treatment of sleep disorders using sleep target modulators

(76) Inventors: Dale Edgar, Wayland, MA (US); David G. Hangauer, East Amherst, NY (US); Harry Jefferson Leighton, Rockport, ME (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/501,855

(22) PCT Filed: Jan. 21, 2003
(86) PCT No.: PCT/US03/01845

§ 371 Date: Jul. 18, 2005

Related U.S. Application Data
(60) Provisional application No. 60/349,912, filed on Jan. 18, 2002.
(60) Provisional application No. 60/357,320, filed on Feb. 15, 2002.

(51) Int. Cl. A61K 31/496; C07D 471/04

(52) U.S. Cl. 514/253.04; 544/362

(57) ABSTRACT

The invention is directed to compositions used for treating sleep disorders. In addition, the invention provides convenient methods of treatment of a sleep disorder. Furthermore, the invention provides methods of treating sleep disorders using compositions that remain active for a discrete period of time to reduce side effects. More specifically, the invention is directed to the compositions and use of ester derivatized trazodone compounds for the treatment of sleep disorders.

### REFERENCE TO RELATED APPLICATION

This application claims priority to pending U.S. Provisional Patent Application Attorney Docket Number HPZ-010-1 (Application No.60/349,912) filed on Jan. 18, 2002, and pending U.S. Provisional Patent Application Attorney Docket Number HPZ-010-2 (Application No.60/357,320) filed on Feb. 15, 2002. This application is also related to pending U.S. Provisional Patent Application Serial No. 60/______ (Attorney Docket Number HPZ-010-3), filed on even date herewith, entitled “Treatment of Sleep Disorders Using Sleep Target Modulators”. The entire content of each of the above-identified applications is hereby incorporated herein by reference.

### BACKGROUND OF THE INVENTION

Difficulties in falling asleep, remaining asleep, sleeping for adequate lengths of time, or abnormal sleep behavior are common symptoms for those suffering with a sleep disorder. A number of sleep disorders, e.g., insomnia or sleep apnea, are described in the online Merck Manual of Medicinal Information.

Current treatment of many sleep disorders include the use of prescription hypnotics, e.g., benzodiazapines, that may be habit-forming, lose their effectiveness after extended use, and metabolize more slowly for certain designated groups, e.g., elderly persons, resulting in persisting medicative effects.

Other, more mild manners of treatment include over-the-counter antihistamines, e.g., diphenhydramine or dimenhydrinate, which are not designed to be strictly sedative in their activity. This method of treatment is also associated with a number of adverse side effects, e.g., persistence of the sedating medication after the prescribed time of treatment, or the so-called “hangover effect”. Many of these side effects result from nonspecific activity in both the periphery as well as the Central Nervous System (CNS) during this period of extended medication.

### SUMMARY OF THE INVENTION

A need exists for the development of new compositions used for the improved treatment of sleep disorders that remain active for a discrete period of time to reduce side effects, such as the “hangover effect.”

Therefore, the invention is directed to compositions used for treating sleep disorders. In addition, the invention provides convenient methods of treatment of a sleep disorder. Furthermore, the invention provides methods of treating sleep disorders using compositions that remain active for a discrete period of time to reduce side effects.

More specifically, the invention is directed to the compositions and use of ester derivatized trazodone compounds for the treatment of sleep disorders.

Thus, in one aspect of the invention, the invention is directed to a method of treating a serotonin receptor associated disorder. The method comprises administering an effective amount of a therapeutic compound to a subject, such that the disorder is treated. Accordingly, the therapeutic compound can have the formula:
[EG]ᵣ-(SP₂)_{q}-[SR]-(SP₁)ₙ-[MR]
wherein SR is a serotonin receptor antagonist, MR is a metabolite reducing moiety that reduces the formation of wake promoting metabolites, EG is an ester group that modifies the half-life of the therapeutic compound, SP₁ and SP₂ are spacer molecules, n, q, and r are independently 0 or 1, and r and q are 0 when MR is the ester group.

Another aspect of the invention is a method of treating a serotonin receptor associated disorder, comprising administering to a subject an effective amount of a therapeutic compound, such that the disorder is treated. Accordingly, the therapeutic compound can have the formula:
[SR]-(SP)ₙ-[EG]
wherein SR is a serotonin receptor antagonist, EG is an ester group that modifies the half-life of the therapeutic compound, SP is a spacer molecule, and n is 0 or 1.

In another aspect of the invention, the invention is a method of treating a sleep disorder. The method comprises administering an effective amount of a therapeutic compound, such that the sleep disorder is treated, wherein the compound has a favorable biological property (FBP).

An additional aspect of the invention is a method of treating a sleep disorder. The method comprises administering an effective amount of a therapeutic compound to a subject, such that the sleep disorder is treated. Accordingly, the therapeutic compound is trazodone compound that contains a moiety selected and positioned, such that a wake promoting metabolite is not formed. The therapeutic compound can have the formula:
[EG]ᵣ-(SP₂)_{q}-[TZ]-(SP₁)ₙ-[MR]
wherein TZ is a trazodone compound, MR is a metabolite reducing moiety that reduces the formation of wake promoting metabolites, EG is an ester group that modifies the half-life of the therapeutic compound, SP₁ and SP₂ are spacer molecules, n, q, and r are independently 0 or 1, and r and q are 0 when MR is the ester group that modifies the half-life of the therapeutic compound.

Another aspect of the invention is directed to a method of treating a sleep disorder. The method comprises administering an effective amount of a therapeutic compound to a subject, such that the sleep disorder is treated. Accordingly, the therapeutic compound can have the formula:
[TZ]-(SP)ₙ-[EG]
wherein TZ is a trazodone compound, EG is an ester group that modifies the half-life of the therapeutic compound, SP is a spacer molecule, and n is 0 or 1.

Another aspect of the invention is a method of modulating a serotonin receptor associated disorder target. The method comprises administering an effective amount of a therapeutic compound to a subject, such that the disorder target is modulated, wherein the therapeutic compound comprises the formula:
[EG]ᵣ-(SP₂)_{q}-[SR]-(SP₁)ₙ-[MR]
wherein SR is a serotonin receptor antagonist, MR is a metabolite reducing moiety that reduces the formation of wake promoting metabolites, EG is an ester group that modifies the half-life of the therapeutic compound, SP₁ and SP₂ are spacer molecules, n, q; and r are independently 0 or 1, and r and q are 0 when MR is the ester group.

Another aspect of the invention is a method of modulating a serotonin receptor associated disorder target. The method comprises administering an effective amount of a therapeutic compound to a subject, such that the disorder target is modulated, wherein the therapeutic compound comprises the formula:
[SR]-(SP)ₙ-[EG]
wherein SR is a serotonin receptor antagonist, EG is an ester group that modifies the half-life of the therapeutic compound, SP is a spacer molecule, and n is 0 or 1.

Another aspect of the invention is a method of modulating a sleep disorder target.

The method comprises administering an effective amount of a therapeutic compound to a subject, such that the sleep disorder target is modulated, wherein the therapeutic compound comprises the formula:
[EG]ᵣ-(SP₂)_{q}-[TZ]-(SP₁)ₙ-[MR]
wherein TZ is a trazodone compound, MR is a metabolite reducing moiety that reduces the formation of wake promoting metabolites, EG is an ester group that modifies the s half-life of the therapeutic compound, SP₁ and SP₂ are spacer molecules, n, q, and r are independently 0 or 1, and r and q are 0 when MR is the ester group that modifies the half-life of the therapeutic compound.

An additional aspect of the invention is a method of modulating a sleep disorder target. The method comprises administering an effective amount of a therapeutic compound to a subject, such that the sleep disorder target is modulated, wherein the therapeutic compound comprises the formula:
[TZ]-(SP)ₙ-[EG]
wherein TZ is a trazodone compound, EG is an ester group that modifies the half-life of the therapeutic compound, SP is a spacer molecule, and n is 0 or 1.

Another aspect of the invention is a compound comprising the formula:
[EG]ᵣ-(SP₂)_{q}-[SR]-(SP₁)ₙ-[MR]
wherein SR is a serotonin receptor antagonist, MR is a metabolite reducing moiety that reduces the formation of wake promoting metabolites, EG is an ester group that modifies the half-life of the therapeutic compound, SP₁ and SP₂ are spacer molecules, n, q, and r are independently 0 or 1, and r and q are 0 when MR is the ester group.

In an additional aspect, the invention is a compound comprising the formula:
[SR]-(SP)ₙ-[EG]
wherein SR is a serotonin receptor antagonist, EG is an ester group that modifies the half-life of the therapeutic compound, SP is a spacer molecule, and n is 0 or 1.

One aspect of the invention is a compound comprising the formula:
[EG]ᵣ-(SP₂)_{q}-[TZ]-(SP₁)ₙ-[MR]
wherein TZ is a trazodone compound, MR is a metabolite reducing moiety that reduces the formation of wake promoting metabolites, EG is an ester group that modifies the half-life of the therapeutic compound, SP₁ and SP₂ are spacer molecules, n, q, and r are independently 0 or 1, and r and q are 0 when MR is the ester group that modifies the half-life of the therapeutic compound.

A further aspect of the invention is a compound comprising the formula:
[TZ]-(SP)ₙ-[EG]
wherein TZ is a trazodone compound, EG is an ester group that modifies the half-life of the therapeutic compound, SP is a spacer molecule, and n is 0 or 1.

Another aspect of the invention is a pharmaceutical composition comprising a therapeutic compound as prepared according to the methodology of this invention, and a pharmaceutically acceptable carrier.

### DETAILED DESCRIPTION OF THE INVENTION

The invention is directed to compositions used for treating sleep disorders. In addition, the invention provides convenient methods of treatment of a sleep disorder. Furthermore, the invention provides methods of treating sleep disorders using compositions that remain active for a discrete period of time to reduce side effects. More specifically, the invention is directed to the compositions and use of ester derivatized trazodone compounds for the treatment of sleep disorders.

### METHODS OF THE INVENTION

One embodiment of the invention is a method of treating a serotonin receptor associated disorder. The method comprises administering an effective amount of a therapeutic compound to a subject, such that the disorder is treated. Accordingly, the therapeutic compound can have the formula:
[EG]ᵣ-(SP₂)_{q}-[SR]-(SP₁)ₙ-[MR]
wherein SR is a serotonin receptor antagonist, MR is a metabolite reducing moiety that reduces the formation of wake promoting metabolites, EG is an ester group that modifies the half-life of the therapeutic compound, SP₁ and SP₂ are spacer molecules, n, q, and r are independently 0 or 1, and r and q are 0 when MR is the ester group. In certain embodiments, the disorder is a sleep disorder.

The language “serotonin receptor antagonist” or “SR” is intended to include antagonists for the receptors for serotonin or 5-HT (5-hydroxytryptamine), i.e., compounds that inhibit the activity of the serotonin receptor and agents that down-regulate (i.e., inhibit) the synthesis or production of the serotonin receptor.

The language “serotonin receptor” is intended to include receptors for serotonin or 5-HT (5-hydroxytryptamine). In certain embodiments of the invention, the receptor is the 5-HT₂ receptor, which belongs to the family of rhodopsin-like signal transducers, distinguished by their seven-transmembrane configuration and their functional linkage to G-proteins. While all the receptors of the serotonin type are recognized by serotonin, they are pharmacologically distinct and are encoded by separate genes. These receptors, known as subtypes, are generally coupled to different second messenger pathways that are linked through guanine-nucleotide regulatory (G) proteins. In certain embodiments, 5-HT₂ receptors activate phospholipase C pathways, stimulating breakdown of polyphosphoinositides.

The 5-HT₂ subfamily—is divided into three receptor subtypes: 5-HT_{2A}, 5-HT_{2B}, and 5-HT_{2C}. The human 5-HT_{2C} receptor was first isolated and cloned in 1987, and the human 5-HT_{2A} receptor was first isolated and cloned in 1990. These two receptors are thought to be the site of action of hallucinogenic drugs. Additionally, antagonists to the 5-HT_{2A} and 5-HT_{2C} receptors are believed to be useful in treating depression, anxiety, psychosis and eating disorders.

In specific embodiments of the invention, the serotonin receptor is a 5-HT_{2A} receptor. In certain embodiments, the 5-HT_{2A} receptor is a specific receptor, which has low affinity for other 5-HT receptor subtypes. Alternatively, the 5-HT_{2A} receptor is a general 5-HT_{2A} receptor, which has a significant affinity to two or more 5-HT receptor subtypes.

The language “a serotonin receptor associated disorder” is intended to include any disorder that is associated with the 5-HT receptor. In certain embodiments of the invention, the disorder is associated with the 5-HT₂ receptor, e.g., the 5-HT_{2A} receptor. Serotonin is thought to play a role in processes related to learning and memory, sleep, thermoregulation, mood, motor activity, pain, sexual and aggressive behaviors, appetite, neurodegenerative regulation, and biological rhythms. Moreover, serotonin has been linked to pathophysiological conditions such as anxiety, depression, obsessive-compulsive disorders, schizophrenia, suicide, autism, migraine, emesis, alcoholism and neurodegenerative disorders.

Exemplary 5-HT₂ antagonists which are considered to be within the scope of the present invention include, but are not limited to adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, triazolam, trepipam, tricetamide, triclofos, trifluoperazine, trimetozine, trimipramine, uldazepam, valproate, venlafaxine, zaleplon, zolazepam, zolpidem, and salts thereof, and combinations thereof.

Another embodiment of the invention is a method of treating a serotonin receptor associated disorder, comprising administering to a subject an effective amount of a therapeutic compound, such that the disorder is treated. Accordingly, the therapeutic compound can have the formula:
[SR]-(SP)ₙ-[EG]
wherein SR is a serotonin receptor antagonist, EG is an ester group that modifies the half-life of the therapeutic compound, SP is a spacer molecule, and n is 0 or 1.

Another aspect of the invention is a compound comprising the formula:
[EG]ᵣ-(SP₂)_{q}-[SR]-(SP₁)ₙ-[MR]
wherein SR is a serotonin receptor antagonist, MR is a metabolite reducing moiety that reduces the formation of wake promoting metabolites, EG is an ester group that modifies the half-life of the therapeutic compound, SP₁ and SP₂ are spacer molecules, n, q, and r are independently 0 or 1, and r and q are 0 when MR is the ester group.

In an additional aspect, the invention is a compound comprising the formula:
[SR]-(SP)ₙ-[EG]
wherein SR is a serotonin receptor antagonist, EG is an ester group that modifies the half-life of the therapeutic compound, SP is a spacer molecule, and n is 0 or 1.

Another aspect of the invention is a method of modulating a serotonin receptor associated disorder target. The method comprises administering an effective amount of a therapeutic compound to a subject, such that the disorder target is modulated, wherein the therapeutic compound comprises the formula:
[EG]ᵣ-(SP₂)_{q}-[SR]-(SP₁)ₙ-[MR]
wherein SR is a serotonin receptor antagonist, MR is a metabolite reducing moiety that reduces the formation of wake promoting metabolites, EG is an ester group that modifies the half-life of the therapeutic compound, SP₁ and SP₂ are spacer molecules, n, q, and r are independently 0 or 1, and r and q are 0 when MR is the ester group.

Another aspect of the invention is a method of modulating a serotonin receptor associated disorder target. The method comprises administering an effective amount of a therapeutic compound to a subject, such that the disorder target is modulated, wherein is the therapeutic compound comprises the formula:
[SR]-(SP)ₙ-[EG]
wherein SR is a serotonin receptor antagonist, EG is an ester group that modifies the half-life of the therapeutic compound, SP is a spacer molecule, and n is 0 or 1.

Another embodiment of the invention is a method of treating a sleep disorder. The method of treating comprises administering to a subject an effective amount of a therapeutic compound, such that the sleep disorder is treated.

The language “sleep disorder,” is art recognized and includes disorders or states that affect a subjects ability to sleep, and which are treatable by the compounds described herein. Sleep disorders generally involve disturbances of sleep that affect a subject's ability to fall and/or stay asleep, and involve sleeping too little, too much or resulting in abnormal behavior associated with sleep. Examples include, but are not limited to disturbed biological and circadian rhythms; and sleep disturbances associated with such diseases as neurological disorders, neuropathic pain and restless leg syndrome; allergies; tolerance to narcotics or withdrawal from narcotics; sleep apnea; narcolepsy, insomnia; Disorders of Initiating and Maintaining Sleep (insomnias) (“DIMS”) which can arise from psychophysiological causes, as a consequence of psychiatric disorders particularly related to anxiety), from drugs and alcohol use and abuse (particularly during withdrawal stages), childhood onset DIMS, nocturnal myoclonus and restless legs and non specific REM disturbances as seen in aging; parasomnia; jet-lag syndrome; hypersomnia, sleep apnea, REM sleep interruptions, shift workers' sleep disturbances, dysomnias, night terror, insomnias associated with depression or with emotional/mood disorders, as well as sleep walking and enuresis, as well as sleep disorders which accompany aging, mental and physical disorders associated with travel across time zones and with rotating shift-work schedules, or syndromes such as fibromyalgia that are manifested by non-restorative sleep and muscle pain or sleep apnea which is associated with respiratory disturbances during sleep; and drug abuse. Difficulties in falling asleep, remaining asleep, sleeping for adequate lengths of time, or abnormal sleep behavior are common symptoms for those suffering with a sleep disorder. A number of sleep disorders, e.g., insomnia or sleep apnea, are described in the online Merck Manual of Medicinal Information.

The administration to a subject of an appropriate amount of a compound of the invention, is useful, for example, in the prevention or treatment of the following conditions to achieve chronobiological effects and/or to alleviate circadian rhythm phase disturbances: disorders of the sleep-wake schedule; jet lag; shift work; people who have a maladaption to work and off-work schedules; medical residents, nurses, firemen, policemen or those whose duties require alertness and wakefulness at evening or nighttime hours, or those deprived of sleep for various periods because of their duties or responsibilities; animal workers; athletes who wish to reset their internal clock to a more beneficial time; the infantry, or other members of the armed forces whose duties require extreme levels of alertness and wakefulness, and those who may be sleep deprived in the performance of these duties; submariners, or people confined for research, exploration or industrial purposes below the seas; miners, spelunkers, researchers or those confined beneath the Earth; astronauts in orbit around the Earth, on missions in space to the Earth's moon or to the planets or out of the solar system, or in training for such missions; the blind or sight-impaired or those persons whose ability to distinguish differences in light and dark may be permanently or temporarily impaired; psychiatric patients; insomniacs; the comatose, or those who need to be maintained in a state of unconsciousness for medical, psychiatric or other reasons; residents of the far North or Antarctica, or those persons who live in a climate or climates which possess abnormal amounts of light or darkness; those suffering from seasonal affective disorder (SAD), winter depression, or other forms of depression; the aged; Alzheimer's disease patients, or those suffering from other forms of dementia; patients who require dosages of medication at appropriate times in the circadian cycles; patients suffering from delayed sleep phase syndrome, advanced sleep phase syndrome, or non-24 hr sleep phase syndrome; and patients suffering from primary or secondary insomnia or circadian rhythm-related insomnia. The present invention is useful, for example, in the prevention or treatment of conditions associated with circadian rhythmicity as well as mental and physical disorders associated with travel across time zones and with rotating shift-work schedules.

The language “insomnia” is characterized by difficulty in sleeping or disturbed sleep patterns. Insomnia may be of a primary nature with little apparent relationship to immediate somatic or psychic events, or secondary to some acquired pain, anxiety or depression, and is further described by Mondadori et al. in U.S. Pat. No. 6,277,864.

The terms “treating” or “treatment” include administering a therapeutically effective compound sufficient to reduce or eliminate at least one symptom of the state, disease or disorder, e.g., a sleep disorder. It will be appreciated to those skilled in the art that reference herein to treatment extends to prophylaxis (prevention) as well as the treatment of the noted diseases/disorders and symptoms.

The language “administering” includes delivery to a subject by any means that does not affect the ability of the therapeutic compound to perform is intended function. The therapeutic compound may be administered by any means that sufficiently treats the disorder target. Administration includes, but is not limited to parenteral, enteral, and topical administration. While it is possible for a compound of the present invention to be administered alone, it is preferable to administer the compound as a pharmaceutical composition, which includes compositions that comprise the compounds of the present invention and a pharmaceutically acceptable carrier. In a specific embodiment, the therapeutic compound is administered orally.

Administration also includes the use of an additional modulating factor (AMF) in “combination therapy.” The language “additional modulating factor (AMF)” includes additional factors, such as additional therapeutics or abnormalities in the subject, e.g., a chemical imbalance. It should be understood that the additional modulating factor may be directed to or affect the same or a different disorder target as that being modulated by the compounds of the present invention. The language “combination therapy” includes the co-administration of the modulating compound of the present invention in the presence of an additional modulating factor, e.g., an additional therapeutic agent. Administration of the modulating compound may be first, followed by the other therapeutic agent; or administration of the other therapeutic agent may be first, followed by the modulating, e.g., inhibiting, compound. The other therapeutic agent may be any agent which is known in the art to treat, prevent, or reduce the symptoms of the targeted disorder, e.g., a sleep disorder. Furthermore, the other therapeutic agent may be any agent of benefit to the patient when administered in combination with the administration of a modulating, e.g., inhibiting, compound.

For example, a therapeutic compound of the invention may be administered in conjunction with a variety of commercially-available drugs, including, but not limited to, antimicrobial agents, such as pentamidine, lomefloxacin, metronidazole; fungistatic agents; germicidal agents; hormones; antipyretic agents; antidiabetic agents; bronchodilators, such as aminophylline; antidiarrheal agents, such as diphenoxylate hydrochloride with atropine sulfate; antiarrhythmic agents, such as disopyramide phosphate and bidisomide; coronary dilation agents; glycosides; spasmolytics; antihypertensive agents, such as verapamil and verapamil hydrochloride and their enantiomers, and betaxolol; antidepressants; antianxiety agents; other psychotherapeutic agents, such as zolpidem, cycloserine and milacemide; corticosteroids; analgesics, such as misoprostol with diclofenac; contraceptives, such as ethynodiol diacetate with ethinyl estradiol, and norethynodrel with mestranol; nonsteroidal anti-inflammatory drugs, such as oxaprozen; blood glucose lowering agents; cholesterol lowering agents; anticonvulsant agents; other antiepileptic agents; immunomodulators; antioholinergics; sympatholytics; sympathomimetics; vasodilatory agents; anticoagulants; antiarrhythmics, such as disopyramide or disobutamide; prostaglandins having various pharmacologic activities, such as misoprostol and enisoprost; diuretics, such as spironolactone and spironolactone with hydrochlorothiazide; sleep aids, such as zolpidem tartrate; antihistaminic agents; antineoplastic agents; oncolytic agents; antiandrogens; antimalarial agents,; antileprosy agents; and various other types of drugs. See Goodman and Gilman's The Basis of Therapeutics (Eighth Edition, Pergamon Press, Inc., USA, 1990) and The Merck Index (Eleventh Edition, Merck & Co., Inc., USA, 1989), each of which is incorporated herein by reference.)

The other therapeutic agent may also be a modulating compound. In addition, the compounds of the present invention can also be administered in combination with other known therapies for the target disorder. For example, the trazodone compound may be administered in conjunction with other compounds that are known in the art to be useful for enhancing sleep quality and preventing and treating sleep disorders and sleep disturbances, including compounds known in the art to be useful for suppressing or stimulating melatonin production, such as, melatonergic agents, noradrenergic and serotonergic re-uptake blockers, alpha-1-noradrenergic agonists, monamine oxidase inhibitors, neuropeptide Y agonists or antagonists; neurokinin-1 agonists; substance P; beta-adrenergic blockers and benzodiazepines, such as atenolol; other compounds that are known in the art to be useful for stimulating melatonin production including tricyclic antidepressants and alpha-2-adrenergic antagonists; melatonin precursors such as tryptophan, 5-hydroxytryptophan, serotonin and N-acetylserotonin; as well as melatonin analogs, melatonin agonists and melatonin antagonists, and melatonin, itself. In addition, the trazodone compound may be administered in conjunction with other compounds which are known in the art to be useful for enhancing sleep quality and preventing and treating sleep disorders and sleep disturbances, including e.g., sedatives, hypnotics, anxiolytics, antipsychotics, antianxiety agents, minor tranquilizers, benzodiazepines, barbituates, and the like, as well as admixtures and combinations thereof. The trazodone compound may also be administered in conjunction with the use of physical methods such as with light therapy or electrical stimulation.

In addition, the trazodone compound may be administered in association with therapeutically effective amounts of one or more adjunct active ingredients selected from decongestants, aspirin, (acetylsalicylic acid), acetaminophen, non-steroidal anti-inflammatory drugs (NSAIDs), cough suppressants, and expectorants. Said adjunct ingredients are dosed at levels known to those skilled in the art and as described in the Physicians' Desk Reference. Representative NSAIDs include, but are not limited to, naproxen, ibuprofen, ketoprofen, benoxaprofen, fluribiprofen, fenoprofen, fenbufen, indoprofen, pirprofen, carprofen, oxaprozin, pranoprofen, miroprofen, tioxaprofen, suprofen, albinoprofen, tiaprofenic acid, fluprofen, bucloxic acid, or pharmaceutically acceptable salts thereof.

Furthermore, a compound of the invention also may be administered in conjunction with any one or combination of the commercially-available, over-the-counter or prescription medications, including, but not limited to Avobenzene/padimate-O, ACCUPRIL® tablets (quinapril hydrochloride), Accutane capsules (isotretinoin), Achromycin V capsules (the monohydrochloride of (4S-(4α,4a.alpha.,5aα,6β, 12aα,))-4-(dimethylamino)-1,4,4a,5,5a,6,11,12a-octBPydro-3,6,10,12,12a-pentBPydroxy-6-methyl-1,11-dioxo-2-naphthacenecarboxamide), Actifed cough syrup (codeine phosphate, triprolidine hydrochloride and pseudoephedrine hydrochloride), Aldactazide tablets (spironolactone and hydrochlorothiazide), ALDOCLOR® tablets (methyldopa and chlorothiazide), Aldoril tablets (methyldopa-hydrochlorothiazide), Alferon® N injection (interferon .alpha-n3 (human leukocyte derived)), ALTACE™ capsules (ramipril), AMBIEN® tablets (zolpidem tartrate), Anafranil capsules (clomipramine hydrochloride), ANAPROX® tablets (naproxen sodium), Ancobon capsules (flucytosine), Ansaid tablets (flurbiprofen), Apresazide capsules (hydralazine hydrochloride and hydrochlorothiazide), Asendin tablets (2-chloro-11-(1-piperazinyl)dibenz(b,f)(1,4)-oxazepine), Atretol™ tablets (carbamazepine), Aureomycin ophthalmic ointment (chlortetracycline hydrochloride), Azo Gantanol® tablets (sulfamethoxazole and phenazopyridine hydrochloride), Azo Gantrisin tablets (sulfisoxazole and phenazopyridine hydrochloride), Azulfidine® tablets and EN-tabs (5-((p-(2-pyridylsulfamoyl)phenyl)-azo)salicylic acid), Bactrim tablets (trimethoprim and sulfamethoxazole), Bactrim I.V. infusion (trimethoprim and sulfamethoxazole), Bactrim pediatric suspension (trimethoprim and sulfamethoxazole), Bactrim suspension (trimethoprim and sulfamethoxazole), Bactrim tablets (trimethoprim and sulfamethoxazole), Benadryl® capsules (diphenhydramine hydrochloride USP), Benadryl® kapseals (diphenhydramine hydrochloride USP), Benadryl® tablets (diphenhydramine hydrochloride USP), Benadryl® parenteral (diphenhydramine hydrochloride USP), Benadryl® steri-vials, ampoules, and steri-dose syringe (diphenhydramine hydrochloride USP), Capoten tablets (captopril), Capozide tablets (captopril-hydrochlorothiazide), Cardizem® CD capsules (diltiazem hydrochloride), Cardizem® SR capsules (diltiazem hydrochloride), Cardizem® tablets (diltiazem hydrochloride), Chibroxin sterile ophthalmic solution (with oral form) (norfloxacin), Children's Advil® suspension (ibuprofen), Cipro® I.V. (ciprofloxacin), Cipro® tablets (ciprofloxacin), Claritin tablets (loratadine), Clinoril tablets (sulindac), Combipres® tablets (clonidine hydrochloride and chlorthalidone), Compazine® injection (prochlorperazine maleate), Compazine® multi-dose vials (prochlorperazine maleate), Compazine® syringes (prochlorperazine maleate), Compazine® spansule capsules (prochlorperazine maleate), Compazine® suppositories (prochlorperazine maleate), Compazine® syrup (prochlorperazine maleate), Compazine® tablets (prochlorperazine maleate), Cordarone tablets (amiodarone hydrochloride), Corzide tablets (nadolol and bendroflumethiazide), Dantrium capsules (dantrolene sodium), Dapsone tablets (4-4′diaminodiphenylsulfone), DAYPRO® caplets (oxaproxin), Declomycin tablets (demeclacycline or (4S-(4α,4aα,5aα,6β,12aα))-7-Chloro-4-dimethyl amino)-1,4,4a,5,5a,6,11,12a-octBPydro-3,6,10,12,12a-pentBPydroxy-1,11-dioxo-2-naphthacenecarboxamide monohydrochloride), DECONAMINE® capsules (chlorpheniramine maleate and d-psuedoephedrine hydrochloride), DECONAMINE® syrup (chlorpheniramine maleate and d-psudoephedrine hydrochloride), DECONAMINE® tablets (chlorpheniramine maleate and d-psudoephedrine hydrochloride), Depakene capsules (valproic acid), Depakene syrup (valproic acid), Depakote sprinkle capsules (divalproex sodium), Depakote tablets (divalproex sodium), DiaBeta® tablets (glyburide), Diabinese tablets (chlorpropamide), Diamox parenteral (acetazolamide), Diamox sequels (acetazolamide), Diamox tablets (acetazolamide), Dimetane-DC cough syrup (brompheniramine maleate, phenylpropanolamine hydrochloride and codeine phosphate), Dimetane-DX cough syrup (brompheniramine maleate, phenylpropanolamine hydrochloride and codeine phosphate), Dipentum® capsules (olsalazine sodium), Diucardin tablets (hydroflumethiazide), Diupres tablets (reserpine and chlorothiazide), Diuril oral suspension (chlorothiazide), Diuril sodium intravenous (chlorothiazide), Diuril tablets (chlorothiazide), Dolobid tablets (diflunisal), DORYX® capsules (doxycycline hyclate), Dyazide capsules (hydrochlorothiazide and triamterene), Dyrenium capsules (triamterene), Efudex cream (5-fluorouracil), Efuidex solutions (5-fluorouracil), Elavil injection (amitriptyline HCl), Elavil tablets (amitriptyline HCl), Eldepryl tablets (selegiline hydrochloride), Endep tablets (amitriptyline HCl), Enduron tablets (methyclothiazide), Enduronyl Forte tablets (methyclothiazide and deserpidine), Enduronyl tablets (methyclothiazide and deserpidine), Ergamisol tablets (levamisole hydrochloride), Esidrix tablets (hydrochlorothiazide USP), Esimil tablets (guanethidine monosulfate USP and hydrochlorothiazide USP), Etrafon Forte tablets (perphenazine, USP and amitriptyline hydrochloride, USP), Etrafon 2-10 tablets (perphenazine, USP and amitriptyline hydrochloride, USP), Etrafon tablets (perphenazine, USP and amitriptyline hydrochloride, USP), Etrafon-A tablets (perphenazine, USP and amitriptyline hydrochloride, USP), Eulexin capsules (flutamide), Exna tablets (benzthiazide), FUDR injection (floxuridine), Fansidar tablets (N1-(5,6-dinethoxy-4-pyrimidinyl) sulfanilamide (sulfadoxine) and 2,4-diamino-5-(p-chlorophenyl)-6-ethylpyrimidine (pyrimethamine), Feldene capsules (piroxicam), Flexeril tablets (cyclobenzaprine hydrochloride), FLOXIN® I.V. (ofloxacin injection), FLOXINS® tablets (ofloxacin), Fluorouracil injection (5-fluoro-2,4 (1H,3H)-pyrimidinedione), Fulvicin tablets (griseofulvin), Gantanol® suspension (sulfamethoxazole), Gantanol® tablets (sulfamethoxazole), Gantrisin ophthalmic ointaent/solution (sulfisoxazole), Gantrisin pediatric suspension (sulfisoxazole), Gantrisin syrup (sulfisoxazole), Gantrisin tablets (sulfisoxazole), Glucotrol tablets (glipizide), Glynase PresTab tablets (glyburide), Grifuilvin V tablets (griseofulvin), Grifulvin oral suspension (griseofulvin), Gristactin capsules (griseofulvin), Grisactin tablets (griseofulvin), Gris-PEG tablets (griseofulvin), Grivate tablets (griseofulvin), Grivate suspension (griseofulvin), Haldol Decanoate 50 injection aloperidol decanoate), Haldol Decanoate 100 injection (haloperidol decanoate), Haldol tablets (haloperidol decanoate), Hibistat germicidal hand rinse (chlorhexidine gluconate), HISMANAL® tablets (astemizole), HydroDIURIL tablets (hydrochlorothiazide), Hydromox tablets (quinethazone), Hydropres tablets (reserpine and hydrochlorothiazide), Inderide® tablets (propranolol hydrochloride and hydrochlorothiazide), Inderides capsule® (propranolol hydrochloride and hydrochlorothiazide), Intal inhaler (cromolyn sodium), Intron A injection (recombinant interferon .alpha.-2b), Lamprene capsules (clofazimine), Lasix oral solution (furosemide), Lasix tablets (furosemide), Lasix injection (furosemide), Limbitrol tablets (chlordiazepoxide and amitriptyline hydrochloride), Lodine capsules (etodolac), Lopressor HCT tablets (metoprolol tartrate USP and hydrochlorothiazide USP), Lotensin tablets (benazepril hydrochloride), LOZOL® tablets (indapamide), Ludiomil tablets (maprotiline hydrochloride USP), Marplan tablets (isocarboxazid), MAXAQUIN® tablets (lomefloxacin HCl), Maxzide tablets (triamterene USP and hydrochlorothiazide USP), Mellaril® concentrate (thioridazine), Mellarilg tablets (thioridazine), Mellaril-S suspension (thioridazine), Mepergan injection (meperidine hydrochloride and promethazine hydrochloride), Methotrexate tablets (methotrexate), Mevacor tablets (lovastatin), Micronase tablets (glyburide), Minizide capsules (prazosin hydrochloride and polythiazide), Minocin intravenous ((4S-(4α,4aα,5aα,12aα))-4,7-bis(dimethylamino)-1,4,4a,5,5a,6,11,12a-octBPydro-3,10,12,12a-tetrBPydroxy-1,11-dioxo-2-naphthace necarboxamide monohydrochloride), Minocin oral suspension ((4S-(4α,4aα.,5aα,12aα))-4,7-bis(dimethylamino)-1,4,4a,5,5a,6,11,12a-octBPydro-3,10,12,12a-tetrBPydroxy-1,11-dioxo-2-naphthacenecarboxamide monohydrochloride), Minocin capsules ((4S-(4.alpha.,4a.alpha,5a.alpha,12a.alpha.))-4,7-bis(dimethylamino)-1,4,4a,5,5a,6,11,12a-octBPydro-3,10,12,12a-tetrBPydroxy-1,11-dioxo-2-naphthace necarboxamide monohydrochloride), Moduretic tablets (amiloride HCl-hydrochlorothiazide), Monodox® capsules (doxycycline monohydrate), Monopril tablets (fosinopril sodium), Children's Motrin liquid suspension (ibuprofen), Motrin tablets (ibuprofen), Mykrox tablets (metolazone), NAPROSYN® suspension (naproxen), NAPROSYN® tablets (naproxen), Navane capsules (thiothixene), Navane intramuscular (thiothixene), NegGram caplets (nalidixic acid), NegGram suspension (nalidixic acid), Neptazane tablets (methazolamide), Nipent injection (pentostatin), Normodyne tablets (labetalol HCl), NOROXIN tablets (norfloxacin), Norpramin tablets (desipramine hydrochloride USP), oretic tablets (hydrochlorothiazide), Oreticyl Forte tablets (hydrochlorothiazide and deserpidine), Orinase tablets (tolbutamide), Ornade capsules (phenylpropanolamine hydrochloride and chlorpheniramine maleate), Orudis capsules (ketoprofen), Oxsoralen lotion (methoxypsoralen), PBZ tablets (tripelennamine hydrochloride USP), PBZ-SR tablets (tripelennamine hydrochloride USP), pHisoHex topical emulsion (hexachlorophene), P & S PLUS® topical tar gel (crude coal tar), Pamelor® capsules (nortriptyline HCl), Pamelor® solution (nortriptyline HCl), Paxil tablets (paroxetine hydrochloride), Pediazole oral suspension (erythromycin ethylsuccinate, USP and sulfisoxazole acetyl, USP), Penetrex™ tablets (enoxacin), Pentasa capsules (mesalamine), Periactin syrup (cyproheptadine HCl), Periactin tablets (cyproheptadine HCl), Phenergan tablets (promethazine hydrochloride), Phenergan injection (promethazine hydrochloride), Phenergan suppositories (promethazine hydrochloride), Phenergan syrup (promethazine hydrochloride), Polytrim® ophthalmic solution (trimethoprim sulfate and polymyxin B sulfate), Pravachol (pravastatin sodium), Prinivil® tablets (lisinopril, MSD), Prinzide tablets (lisinopril-hydrochlorothiazide), Prolixin elixir (fluphenazine hydrochloride), Prolixin enanthate (fluphenazine hydrochloride), Prolixin injection (fluphenazine hydrochloride), Prolixin oral concentrate (fluphenazine hydrochloride), Prolixin tablets (fluphenazine hydrochloride), ProSom tablets (estazolam), Prozac® oral solution (fluoxetine hydrochloride), Prozac® oral Pulvules® (fluoxetine hydrochloride), Pyrazinamide tablets (pyrazinamide), QUINAGLUTE® tablets (quinidine gluconate), Quinidex tablets (quinidine sulfate), Relafen tablets (nabumetone), Ru-Tuss II capsules (chlorpheniramine maleate and phenylpropanolamine hydrochloride), Seldane tablets (terfenadine), Septra tablets (trimethoprim and sulfamethoxazole), Septra suspension (trimethoprim and sulfamethoxazole), Septra I.V. infusion (trimethoprim and sulfamethoxazole), Septra tablets (trimethoprim and sulfamethoxazole), Ser-Ap-Es tablets (reserpine USP, hydralazine hydrochloride USP and hydrochlorothiazide USP), Sinequan capsules (doxepin HCl), Solganal injection (aurothioglucose, USP), Stelazine concentrate (trifluoperazine hydrochloride), Stelazine injection (trifluoperazine hydrochloride), Stelazine tablets (trifluoperazine hydrochloride), Surmontil capsules (trimipramine maleate), SYMMETREL capsules and syrup (amantadine hydrochloride), Taractan concentrate (chlorprothixene), Taractan injectable (chlorprothixene), Taractan tablets (chlorprothixene), TAVIST® syrup (clemastine fumarate, USP), TAVIST® tablets (clemastine fumarate, USP), TAVIST®-1 12 hour relief medicine (clemastine fumarate, USP), TAVIST®-D 12 hour relief medicine (clemastine fumarate, USP), Tegretol Tablets (carbamazepine USP), Tegretol suspension (carbamazepine USP), Temaril tablets (trimeprazine tartrate), Temaril syrup (trimeprazine tartrate), Temaril capsules (trimeprazine tartrate), TENORETIC® tablets (atenolol and chlorthalidone), Terramycin intramuscular solution (oxytetracycline), Thiosulfil Forte tablets (sulfamethizole), Thorazine ampuls (chlorpromazine hydrochloride), Thorazine concentrate (chlorpromazine hydrochloride), Thorazine multi-dose vials (chlorpromazine hydrochloride), Thorazine capsules (chlorpromazine hydrochloride), Thorazine suppositories (chlorpromazine hydrochloride), Thorazine syrup (chlorpromazine hydrochloride), Thorazine tablets (chlorpromazine hydrochloride), Timolide tablets (timolol maleate-hydrochlorothiazide), Tofranil ampuls (imipramine hydrochloride USP), Tofranil tablets (imipramine hydrochloride USP), Tofranil capsules (imipramine hydrochloride USP), Tolinase tablets (tolazamide), Triaminic Expectorant DH (phenylpropanolamine hydrochloride and guaifenesin), Triaminic oral infant drops (phenylpropanolamine hydrochloride, pheniramine maleate and pyrilamine maleate), Triavil tablets (perphenazine-amitriptyline HCl), Trilafon concentrate (perphenazine USP), Trilafon injection (perphenazine USP), Trilafon tablets (perphenazine, USP), Trinalin tablets (azatadine maleate, USP, and pseudoephedrine sulfate, USP), Vaseretic tablets (enalapril maleate-hydrochlorothiazide), Vasosulf opthalmic solution (sulfacetamide sodium-phenylephrine hydrochloride), Vasotec I.V. (enalapril maleate), Vasotec tablets (enalapril maleate), Velban® vials (vinblastine sulfate, USP), Vibramycin capsules (doxycycline monohydrate), Vibramycin intravenous (doxycycline monohydrate), Vibramycin oral suspension (doxycycline monohydrate), Vibra-Tabs tablets (oxytetracycline), Vivactil tablets (protriptyline HCl), Voltaren tablets (diclofenac sodium), X-SEB T® shampoo (crude coal tar), Zaroxolyn tablets (metolazone), ZESTORETIC® oral (lisinopril and hydrochlorothiazide), ZESTRIL® tablets (lisinopril), ZITHROMAX™ capsules (azithromycin), Zocor tablets (simvastatin), ZOLOFT® tablets (sertraline hydrochloride) and others.

The term “pharmaceutically acceptable carrier” include a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine by molding a mixture of the powdered compound moistened with an inert liquid diluent

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacilying agents and may be of a composition that releases the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art. For example, such inert diluents, include but are not limited to, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrrhydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising. For example, a formulation of the invention may be prepared from cocoa butter, polyethylene glycol, a suppository wax or a salicylate, which is solid at room temperature, but liquid at body temperature, and will, therefore, melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders, which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally; and are of course given by forms suitable for each administration route. For example, the preparations are administered in tablets or capsule form, by injection, inhalation, eye lotion, ointment, suppository, etc. administration by injection, infusion or inhalation; topical by lotion or ointment; and rectal by suppositories. Oral administration is preferred.

The terms “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subaracinoid, intraspinal and intrastemal injection and infusion.

The terms “systemic administration,” “administered systematically,” “peripheral administration” and “administered peripherally” as used herein mean the administration of a compound, drug or other material other than directly into the central nervous system, for example, subcutaneous administration, such that it enters the patient's system and thus, is possibly subject to metabolism and other like processes.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The regimen of administration can affect what constitutes an effective amount. The disorder target modulators, e.g., sleep disorder target modulators, can be administered to the subject either prior to or after the onset of a sleep disorder associated state. Further, several divided dosages, as well as staggered dosages, can be administered daily or sequentially, or the dose can be continuously infused, or can be a bolus injection. Further, the dosages of the disorder target modulators, e.g., sleep disorder target modulators, compound(s) can be proportionally increased or decreased as indicated by the exigencies of the therapeutic or prophylactic situation.

The language “subject” includes animals (e.g., mammals, e.g., cats, dogs, horses, pigs, cows, sheep, rodents, rabbits, squirrels, bears, primates (e.g., chimpanzees, gorillas, and humans) which are capable of suffering from a sleep associated disorder.

The language “therapeutically effective amount” of the compound is that amount necessary or sufficient to treat or prevent a state associated with a disorder, e.g., sleep disorder. The effective amount can vary depending on such factors as the size and weight of the subject, the type of illness, or the particular compound. For example, the choice of the therapeutic compound can affect what constitutes an “effective amount”. One of ordinary skill in the art would be able to study the aforementioned factors and make the determination regarding the effective amount of the therapeutic compound without undue experimentation.

The language “penetrates into the CNS” includes the favorable biological property of a compound of the current invention to pass through, or penetrate, the blood brain barrier (BBB) and enter into the CNS.

The language “therapeutic compound” includes compounds of the invention capable of performing their intended function, e.g., treating sleep disorders and/or modulating sleep targets. The therapeutic compounds of the invention are described in detail herein.

Accordingly, the therapeutic compound can have the formula:
[EG]ᵣ(SP₂)_{q}-[TZ]-(SP₁)ₙ-[MR]
wherein TZ is a trazodone compound, MR is a metabolite reducing moiety that reduces the formation of wake promoting metabolites, EG is an ester group that modifies the half-life of the therapeutic compound, SP₁ and SP₂ are spacer molecules, n, q, and r are independently 0 or 1, and r and q are 0 when MR is the ester group that modifies the half-life of the therapeutic compound.

Moreover, an ester moiety can function as the moiety that inhibits the formation of a wake promoting metabolite, Lie., MR, or a separate group can be used for that purpose. If a separate group is used to inhibit the formation of the wake-promoting metabolite, then an ester moiety can optionally be positioned elsewhere in the drug so as to control its half-life through esterase catalyzed inactivation. However, if an ester group is used to inhibit the formation of a wake promoting metabolite then the same ester group can be, but is not necessarily, used to control the half life of the drug.

The language “trazodone compound”, or “TZ” is intended to include trazodone or analogs thereof The trazodone analogs include, but are not limited to, trazodones containing substituents that do not significantly effect the analog's ability to perform its intended function.

The language “metabolism reducing moiety”, or “MR” is a moiety that provides the ability to reduce the metabolism of the therapeutic compound such that there is a reduction in the wake promoting metabolites formed. Alternatively, MR can be a moiety that modifies the activity of the metabolite. Examples include functional moieties, e.g., esters or alkyl groups, selected and positioned within the therapeutic drug to provide the ability for a reduction in the wake promoting metabolites formed. In certain embodiments, the MR provides the ability to modulate the activity of the drug, e.g., half-life. In certain embodiments of the invention, the metabolism reducing moiety is an ester group, EG. Alternatively, in particular embodiments of the invention the MR is alkyl, e.g., cyclopropyl or gem-dimethyl, as depicted below in Table 2.

The language “wake promoting metabolite” is intended to include a metabolite of the therapeutic compound, produced in vivo that reduces the therapeutic effect on the sleep disorder. In certain embodiments, the wake promoting metabolite is meta-chlorophenylpiperazine (m-CPP).

The language “ester group” or “EG” are used interchangeably and are intended to include an organic ester functionality that is selected and positioned within the compound providing the ability to modulate the activity or modify the properties of the corresponding therapeutic compound, e.g., half-life or metabolite formation. In certain embodiments, the EG modifies the half-life of the therapeutic compound and/or reduces the formation of wake promoting metabolites. The organic ester group may be terminal, e.g., a substituent, or internal. The carboxylate of the ester may be oriented from left to right or from right to left, e.g., a reverse ester. Examples of esters of the current invention include, but are not limited to hydrocarbons and perfluorocarbons. In a preferred embodiment, the hydrocarbon possesses 1 to 20 carbons. In certain embodiments, the hydrocarbon can be linear, branched, cyclic, aromatic, or a combination of saturated or unsaturated aliphatic and aromatic groups, which are optionally substituted with O, N, S, and/or halogens and may additionally include a center of chirality. In particular embodiments, the ester can be an n-propyl, an isopropyl, a t-butyl, an isobutyl, a cyclopentyl, a cyclohexyl, a cycloheptyl, and a benzyl group.

In particular embodiments, the activity of the drug, e.g., half-life, of the therapeutic drug is modulated by controlling the rate of hydrolysis of the ester group by selection and positioning of steric bulk near the ester carbonyl of the ester group, or by the incorporation of electron withdrawing or donating moieties into, or adjacent to, the ester. In certain embodiments, the steric bulk is provided by the selection of a bulky ester group. In alternative embodiments the steric bulk is provided by substitution selected and positioned on the TZ moiety near the carbonyl of the ester group.

The language “bulky ester” is intended to include an ester that has sufficient steric properties such that the rate of hydrolysis of the therapeutic compound is modulated, e.g., reduced, such that the activity of the therapeutic compound is modified, e.g., the length of activity is increased (i.e., the half-life of the therapeutic compound is increased). Examples of bulky ester groups are depicted in Table 1.

TABLE 1
Bulky Ester Groups For H1 Antagonists

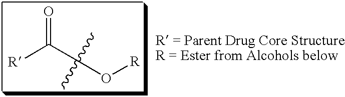

Type A:

| | | |
|---|---|---|
| 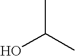 | 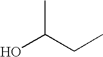 | 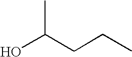 |

Type B:

| | |
|---|---|
| 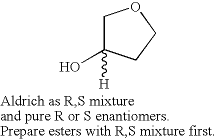 | 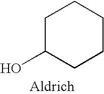 |
| 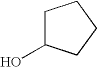 | 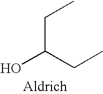 |
| 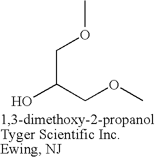 | 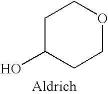 |
| 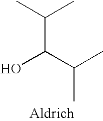 | 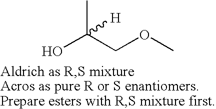 |
| 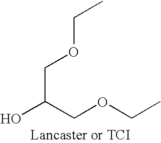 | 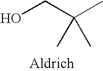 |

The language “hydrocarbon” includes substituted or unsubstituted alkyl, alkenyl, alkynyl, or aryl moieties. The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, the term alkyl includes both “unsubstituted alkyls” and “substituted alkyls”, the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl (benzyl)). The term “alkyl” also includes the side chains of natural and unnatural amino acids.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond.

For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including aLkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “acyl” includes compounds and moieties that contain the acyl radical (CH₃CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthyl carboxy, etc.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group that is bound to at least one alkyl group and at least one aryl group. The term “alkaminoalkyl” refers to an alkyl, alkenyl, or alkynyl group bound to a nitrogen atom that is also bound to an alkyl group.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl, or alkynyl groups bound to an amino group bound to a carboxy group. It includes arylaminocarboxy groups that include aryl or heteroaryl moieties bound to an amino group which is bound to the carbon of a carbonyl or thiocarbonyl group. The terms “alkylaminocarboxy,” “alkenylaminocarboxy,” “alkynylaminocarboxy,” and “arylaminocarboxy” include moieties wherein alkyl, alkenyl, alkynyl and aryl moieties, respectively, are bound to a nitrogen atom which is in turn bound to the carbon of a carbonyl group.

The term “carbonyl” or “carboxy” includes compounds and moieties that contain a carbon connected with a double bond to an oxygen atom. Examples of moieties that contain a carbonyl include aldehydes, ketones, carboxylic acids, amides, esters, anhydrides, etc.

The term “thiocarbonyl” or “thiocarboxy” includes compounds and moieties that contain a carbon connected with a double bond to a sulfur atom.

The term “ether” includes compounds or moieties that contain an oxygen bonded to two different carbon atoms or heteroatoms. For example, the term includes “alkoxyalkyl” which refers to an alkyl, alkenyl, or alkynyl group covalently bonded to an oxygen atom that is covalently bonded to another alkyl group.

The term “thioether” includes compounds and moieties that contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom that is bonded to an alkyl group. Similarly, the term “alkthioalkenyls” and alkthioalkynyls” refer to compounds or moieties wherein an alkyl, alkenyl, or alkynyl group is bonded to a sulfur atom that is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated,” e.g., perfluorinated, generally refers to a moiety, e.g., perfluorocarbons, wherein all hydrogens are replaced by halogen atoms, e.g., fluorine.

The terms “polycyclyl” or “polycyclic radical” refer to two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

In certain embodiments, the ester group or the metabolite reducing moiety (EG or MR) does not substantially effect the biological activity of the therapeutic compound. Alternatively, in certain other embodiments the ester group or the metabolite reducing moiety (EG or MR) significantly effects the biological activity of the therapeutic compound. In one embodiment, the ester group or the metabolite reducing moiety (EG or MR) decreases the biological activity of the therapeutic compound. Alternatively, in an another embodiment of the invention, the ester group or the metabolite reducing moiety (EG or MR) improves the biological activity of the therapeutic compound.

When the ester is a methyl or an ethyl ester, the formulation of the therapeutic compound is formulated to sufficiently treat the target disorder. In addition, formulations of the therapeutic compound can be used to provide controlled in vivo adsorption of the therapeutic compound over a discrete period of time.

In certain embodiments of the invention, the compound containing the metabolism reducing group, e.g., an ester group, is more active as a therapeutic agent for treating disorders than the corresponding compound without the this group, e.g., due to a reduction in the production of the wake-promoting metabolite. In another embodiment of the invention, the compound containing the ester group, is more active as a therapeutic agent for treating disorders than the corresponding acid. In other embodiments, the corresponding acid of the ester is not a therapeutically active agent for treating disorders.

One skilled in the art would recognize that the ester groups, as described above, could be extended to thioesters. Labile amides may also be used in replacement of the ester group, wherein the in vivo hydrolysis would be performed by peptidases in the CNS.

The language “biological activity” includes activity associated with the intended biological function of the compounds of the present invention, e.g., treating a sleep disorder.

The language “modulate a target” or “modulation of a target” includes the act of agonizing or antagonizing a receptor or group of receptors of a target disorder. Thus, a compound that agonizes or antagonizes a receptor or group of receptors is referred to herein as a target modulator, e.g., sleep disorder target modulator.

The language “target modulator” includes compounds or compositions, e.g., pharmaceutical compositions, which are used to modulate a target, e.g., a sleep disorder target.

The term “target” includes a receptor or group of receptors that have been identified as useful point of action for a therapeutic compound, e.g., sleep disorder target.

The language “receptor” includes specific sites of binding or action within a is subject, associated or responsible for the activity of the target disorder, e.g., a 5-HT_{2A} receptor.

The language “group of receptors” includes two or more receptors that may comprise the same receptor type or may comprise two or more receptor types.

The language “compounds that agonize” a receptor is intended to include compounds that induce the activity of the receptor and agents that up-regulate (i.e., induce) the synthesis or production of the receptor.

The language “compounds that antagonize” a receptor, e.g., a 5-HT_{2A} receptor, is intended to include compounds that inhibit the activity of the receptor and agents that down-regulate (i.e., inhibit) the synthesis or production of the receptor.

The terms “modification” or “modifies” include controlling or adjusting physical or chemical parameters, e.g., the half-life, of the therapeutic compound in vivo by changing one or more factors, e.g., the lipophilicity, electronic properties and/or steric size of the metabolite reducing moiety, e.g., ester group.

The language “spacer molecule,” “SP,” “SP,” or “SP₂” includes molecules or moieties that are positioned within the compound to allow the compound to perform its intended function. In certain embodiments, the spacer molecule may be present. Alternatively, in certain other embodiments, the spacer molecule may not be present. In certain embodiments, the spacer molecule may be (CH₂)ₘ, where m is an integer number selected from 1 to 20. In addition, the spacer molecule, e.g., the (CH₂)ₘ linker to an ester or a carboxylic acid group, can be substituted with one or more substituents. In one embodiment, the spacer molecule is mono-substituted. In another embodiment of the invention, the spacer molecule is disubstituted. In particular embodiments, the linkers of the invention may be geminally-dialkylated, e.g., gem-dimethylated; singly substituted with a substituent other than a noncyclic alkyl group, e.g., a heteroatom; or a cyclic substituent wherein one or more of the carbons of the spacer molecule is contained in the ring, e.g., heterocycle (e.g., tetrahydropyran or tetrahydrofuran), or cyclic alkyl, e.g., cyclopropyl. However, the substitution of the spacer molecule is independent of the substitution elsewhere in the molecule.

In particular, the therapeutic compound of the invention may comprise the formula:
[TZ]-(SP)ₙ-[EG]
wherein TZ is a trazodone compound, EG is an ester group that modifies the half-life of the therapeutic compound, SP is a spacer molecule, and n is 0 or 1.

In certain embodiments of the invention, the therapeutic compound has a favorable biological property. In one embodiment of the invention, the invention is a method of treating a sleep disorder. The method comprises administering an effective amount of a trazodone compound, such that the sleep disorder is treated, wherein the trazodone compound has a favorable biological property (FBP).

The language “favorable biological property (FBP)” includes one or more biological properties that allow the compound to perform its intended function in an enhanced manner. Examples of favorable biological properties include but are not limited to induction of a discrete sleep or hypnotic state, activity of the therapeutic compound for a discrete period of time, penetration through the blood brain barrier into the CNS, modulation of the half-life of the therapeutic compound, in vivo hydrolysis of the ester by esterases that allows sequestration of the therapeutic compound in the CNS, reduction of the formation of a wake-promoting metabolite, e.g., m-CPP, an alteration of charge, an alteration of pharmacology-kinetics, an alteration of log P by a value of 0.25 or more, increased receptor selectivity, reduced peripheral half-life, the ability to increase dosage, increased peripheral elimination, increased elimination from the CNS, decreased anti-muscarinic activity, decreased anti-cholinergic, and any combination thereof. It should be understood that the language “FPB” is intended to include a single property or a combination of two or more properties. In particular embodiments of the invention, the therapeutic compound induces a discrete sleep or hypnotic state by penetration into the CNS. In certain embodiments of the invention, the FBP includes increased concentration within the CNS for a discrete period of time as a result of a slower rate of conversion to the corresponding carboxylic acid by in vivo esterase activity within the CNS as compared with the periphery.

In certain embodiments, wherein the therapeutic compound is active for a discrete period of time, the FBP is a reduced ability of the subject to form a tolerance to the therapeutic compound. The language “tolerance” includes the natural tendency of a subject to become less affected by continued administration of a particular therapeutic compound due to repeated exposure to the compound. It should be noted that tolerance is typically increased coincident with the increased time that a compound is present in its active state within the subject. Reduced tolerance would coincide with increased therapeutic effectiveness.

The language “discrete sleep or hypnotic state” includes a state of sedated consciousness that is induced by the presence of active therapeutic compound of the invention, for a defined period of time. This is in contrast to the lingering hangover effect resulting from the existing treatments, e.g., anti-histamines, used for their sedative effect that maintain active drug concentrations for extended periods of time in the periphery.

The language “discrete period of time” includes a defined period of time in which the therapeutic compound is active, and depends upon the physical and reactive properties of the ester group. In one embodiment of the invention, the half-life of the therapeutic compound is 1 to 8 hours. In a preferred embodiment, the half-life of the therapeutic compound is 4 to 6 hours. It should be understood that ranges within these half-life values is intended to be within the scope of this invention.

The term “sequestration” includes having enhanced concentration in the CNS and more rapid elimination from the periphery. The product of hydrolysis can exit the brain by various carboxylate excretion mechanisms, possibly at a slower rate than from the periphery producing a CNS sequestration of the carboxylate for a defined, or discrete, period of time. In one embodiment of the invention, elimination of the hydrolyzed carboxylate-containing metabolite occurs predominately by excretion through the kidneys, due to enhanced polarity of the metabolite, either as the free carboxylate or after Phase II further metabolism. In another embodiment, elimination occurs predominantly by metabolism in the liver, e.g. hydrolysis of the ester followed by glucuronidation, and excretion into the bile. In certain embodiments, the brain assists in the elimination from the CNS though various active transport mechanisms.

Another embodiment of the current invention is a method of modulating a sleep disorder target comprising administering to a subject an effective amount of a therapeutic compound, such that the sleep disorder is treated, wherein the therapeutic compound is as described above and comprises one of the following formulae:
[EG]ᵣ-(SP₂)_{q}-[TZ]-(SP₁)ₙ-[MR], or
[TZ]-(SP)ₙ-[EG]
wherein TZ is a trazodone compound, MR is a metabolite reducing moiety that reduces the formation of wake promoting metabolites, EG is an ester group that modifies the half-life of the therapeutic compound, SP, SP₁ and SP₂ are spacer molecules, n, q, and r are independently 0 or 1, and r and q are 0 when MR is the ester group that modifies the half-life of the therapeutic compound (i.e., EG=MR such that EG also reduces the formation of the wake-promoting metabolite).

Another embodiment of the invention is a sleep disorder target modulator comprising the formula:
[EG]ᵣ-(SP₂)_{q}-[TZ]-(SP₁)ₙ-[MR]
wherein TZ is a trazodone compound, MR is a metabolite reducing moiety that reduces the formation of wake promoting metabolites, EG is an ester group that modifies the half-life of the therapeutic compound, SP₁ and SP₂ are spacer molecules, n, q, and r are independently 0 or 1, and r and q are 0 when MR is the ester group that modifies the half-life of the therapeutic compound.

In another embodiment of the invention, a sleep disorder target modulator comprises the formula:
[TZ]-(SP)ₙ-[EG]
wherein TZ is a trazodone compound, EG is an ester group that modifies the half-life of the therapeutic compound, SP is a spacer molecule, and n is 0 or 1.

In accord with the invention, particular embodiments of the therapeutic compound used for treating disorders are: 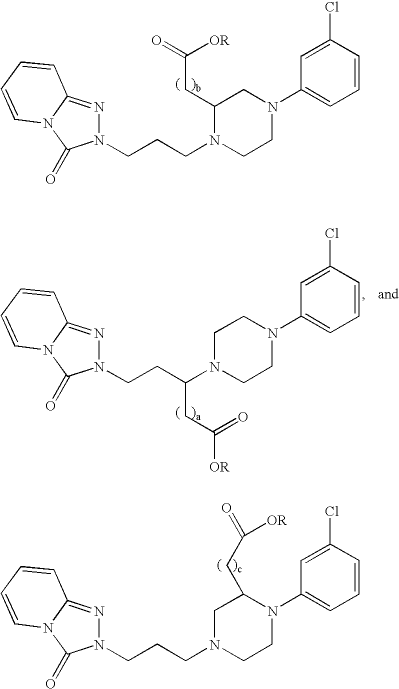
wherein a=0 through 5, b=0 through 5, b=0 through 5, and R is any group which imparts properties to the therapeutic compound to promote penetration into the CNS, reduction of the formation of wake-promoting metabolites, and/or to modify the half-life of the compound. In preferred embodiments of the invention, a=0 or 1; b=0 or 1; and c=0 or 1.

Additional particular embodiments of the therapeutic compound used for treating disorders are: 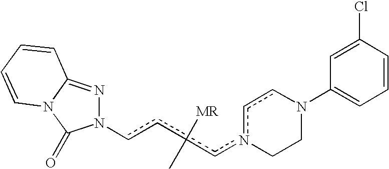
wherein MR is a metabolite reducing moiety that reduces the formation of wake-promoting metabolites. MR is selected and positioned along the dotted line shown above such that the compound is capable of performing its intended function.

In yet another particular embodiment, the therapeutic compound used for treating disorders can have the formula:

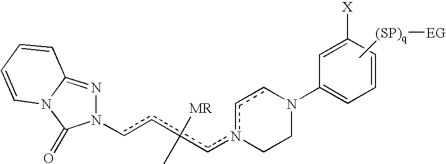

wherein MR is a metabolite reducing moiety that reduces the formation of wake-promoting metabolites, EG is an ester group that modifies the half-life of the therapeutic compound, SP is a spacer molecule, q is 0 or 1, and X is H or Cl, such that MR is selected and positioned along the dotted line shown above such that the compound is capable of performing its intended function. It should be understood that MR can be one or more groups, i.e., functional moieties, which can be attached at multiple positions along the dotted line (e.g., a single MR group may be attached at multiple positions or more than one MR group may be attached at multiple positions). In certain embodiments, MR is alkyl. In particular embodiments, the therapeutic compound of the invention is selected from the compounds listed in Table 2.

TABLE 2

| Structure | Series # |
|---|---|
| 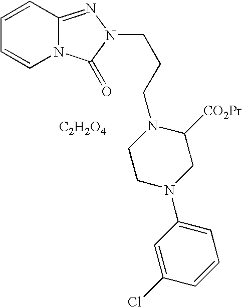 | 18d-oxa- late |
| 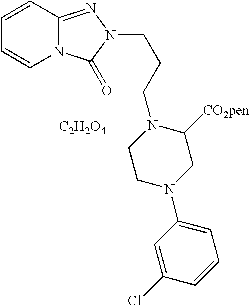 | 18f-oxa- late |
| 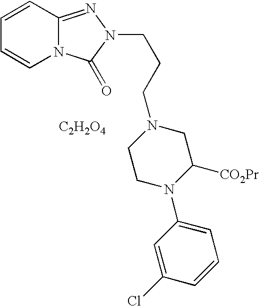 | 19d-oxa- late |
| 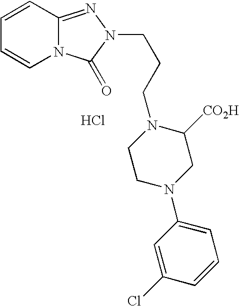 | 18a-HCl |
| 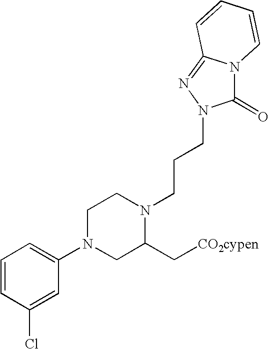 | N/A |
| 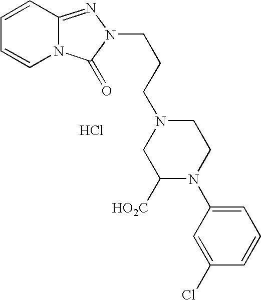 | 19a-HCl |
| 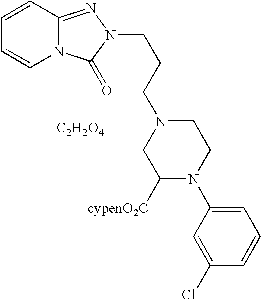 | 19f-oxa- late |
| 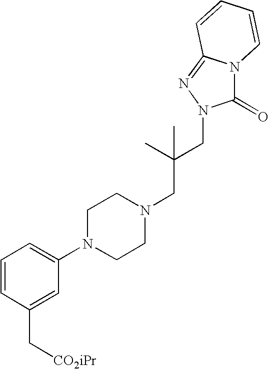 | N/A |
| 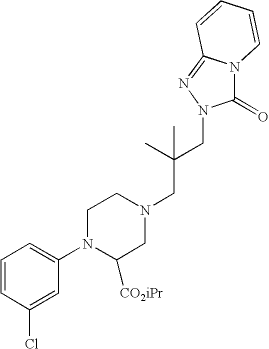 | N/A |
| 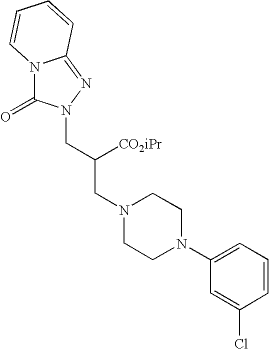 | N/A |
| 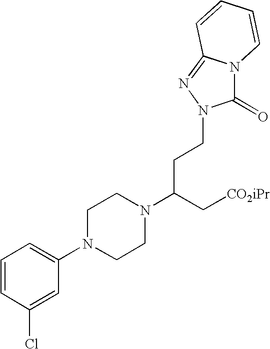 | N/A |
| 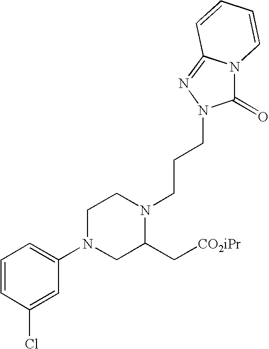 | N/A |
| 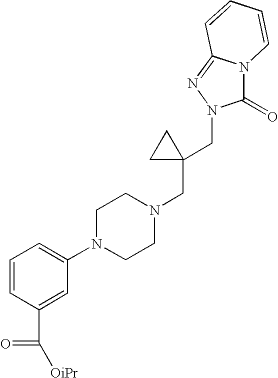 | N/A |
| 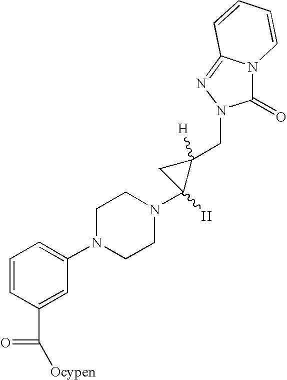 | N/A |
| 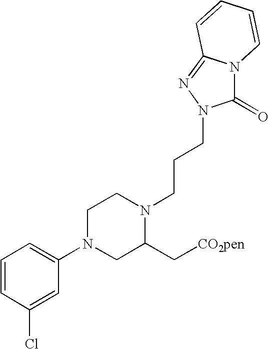 | N/A |
| 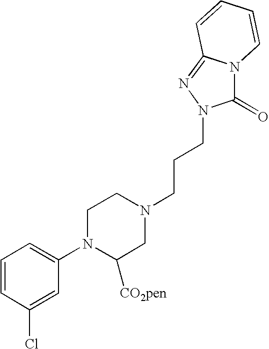 | N/A |
| 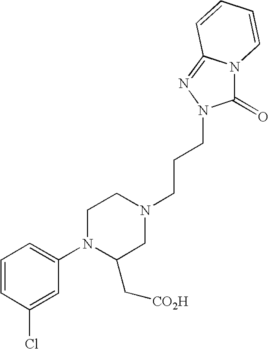 | N/A |
| 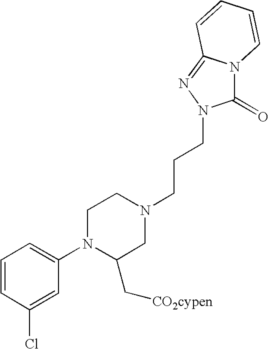 | N/A |
| 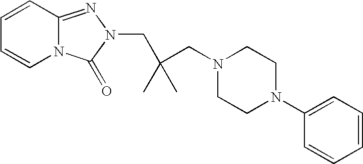 | Free base |
| 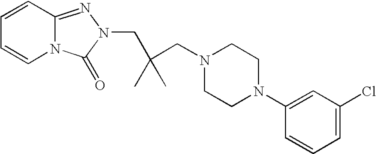 | Free base |
| 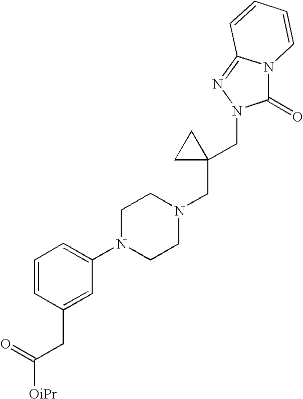 | N/A |
| 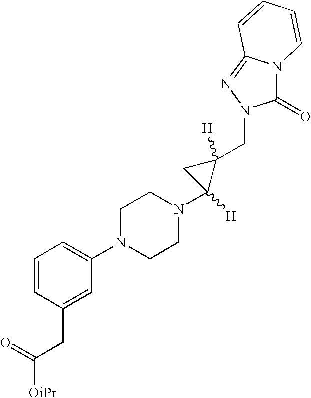 | N/A |
| 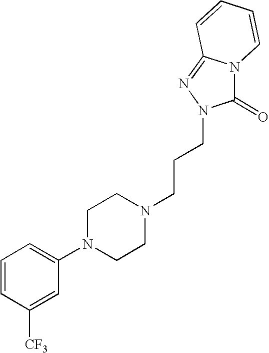 | Free base |
| 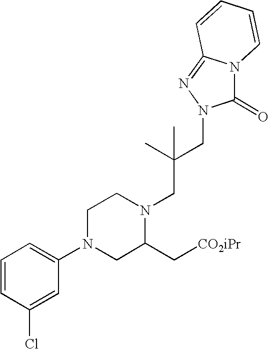 | N/A |
| 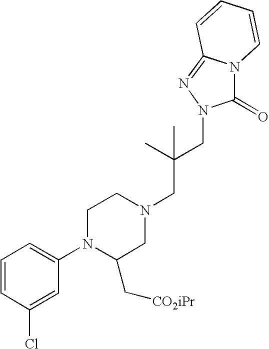 | N/A |
| 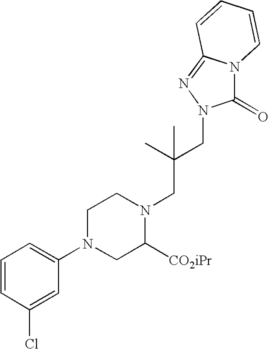 | N/A |
| 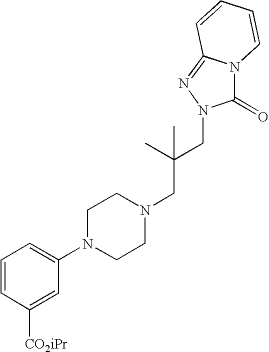 | N/A |
| 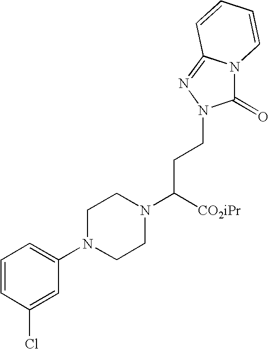 | N/A |
| 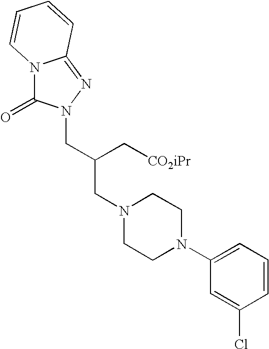 | N/A |
| 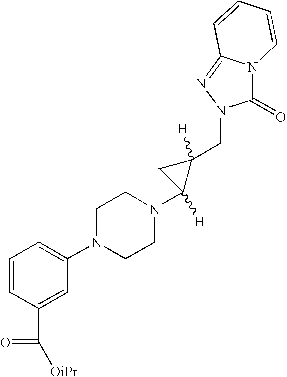 | N/A |
| 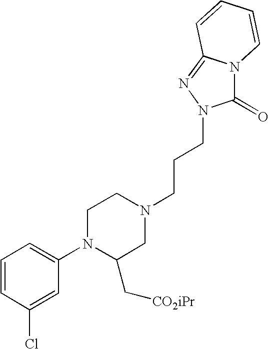 | N/A |
| 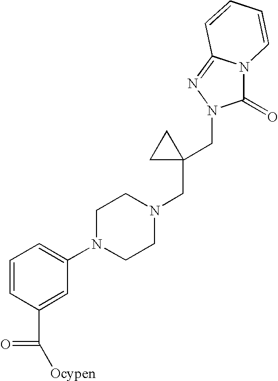 | N/A |
| 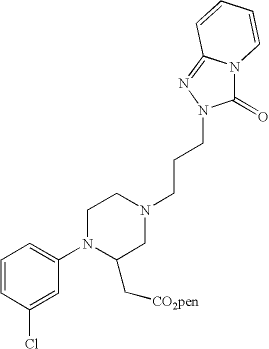 | N/A |
| 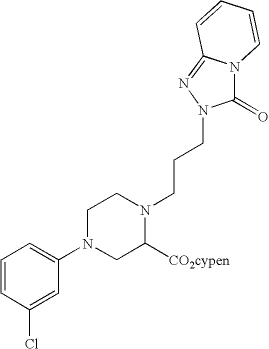 | N/A |
| 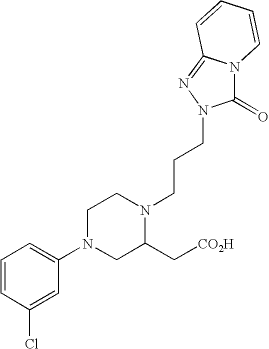 | N/A |

Another embodiment of the invention is a pharmaceutical composition comprising a therapeutic compound as prepared according to the methodology of this invention, and a pharmaceutically acceptable carrier.

In another embodiment, the invention is intended to include any novel compounds described herein.

Additionally, the compounds described above are intended to include analogs containing art-recognized substituents that do not significantly effect the analog's ability to perform its intended function. Furthermore, any novel synthesis of the compounds of the invention described herein, is also intended to be included within the scope of the present invention.

Assays can be used to design and/or select compounds useful within the present invention. The SCORE method, described in Example 2, would be an example of such an assay. Multiple assay components, such as total sleep time, cumulative nonREM sleep profile, maximum nonREM sleep bout length, average nonREM sleep bout length, nonREM sleep time, nonREM onset of action profile, sleep latency, REM sleep time, REM sleep bout length, cumulative REM sleep profile, maximum wake bout length, average wake bout length, locomotor activity, locomotor activity intensity, body temperature, and drinking are used to define compounds that would be useful in the present invention. For example, in determining therapeutic compounds that would be useful as sedatives or wake-promoting compounds, all of the components listed above would be used in determining a preferred therapeutic compound. Antidepressant therapeutic compounds would use the components of total sleep time, cumulative nonREM sleep profile, maximum nonREM sleep bout length, REM sleep time, REM sleep bout length, locomotor activity, locomotor activity intensity, and body temperature for determination of preferred therapeutic compounds.

### EXEMPLIFICATION OF THE INVENTION

The invention is further illustrated by the following examples that should not be construed as limiting. Compounds described herein may be obtained through art recognized synthesis strategies.

### EXAMPLE 1

Several synthetic protocols for compounds of the invention and intermediates thereto are described below and depicted in the corresponding schemes, shown below.

Compound 1. Compound 1 was synthesized following the similar procedure reported by Lis, R.; Marisca, A. J. A Convenient Synthesis of N-Aryl-N′-Benzyl-1,2-Ethanediamines. Synth. Commun. 1988, 18, 45-50.

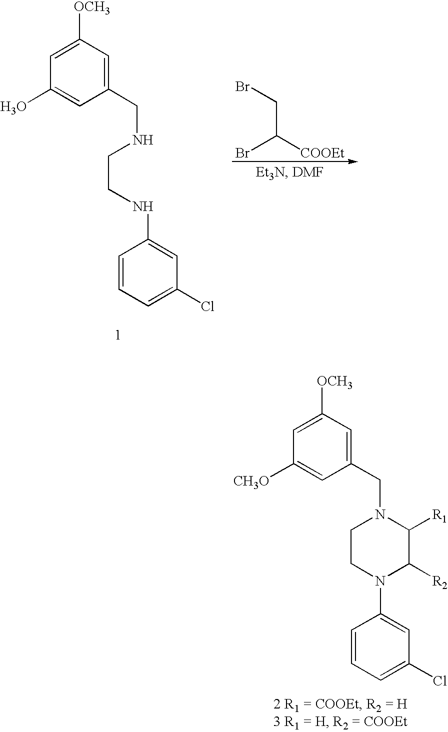

Compound 2 and 3. Compound 1 (19.5 g, 60.93 mmol) and ethyl 2,3-dibromopropionate (30.2 g, 117.36 mmol) were dissolved in DMF (55 mL). Triethylamine (32.5 mL, 234.72 mmol) was added to give a slurry, which then was heated in an oil bath at 110° C. for 17 h. The reaction was cooled to room temperature and 1 N NaOH (80 mL) was added. The resulting solid was collected by filtration and crystallized from 2-propanol to give 9.2 g of compound 3. The mother liquor was then concentrated and purified by column chromatography (silica) to give compound 2 (4.1 g). Compound 2 and 3 were confirmed by ¹H-NMR, ¹³C-NMR and LC-MS.

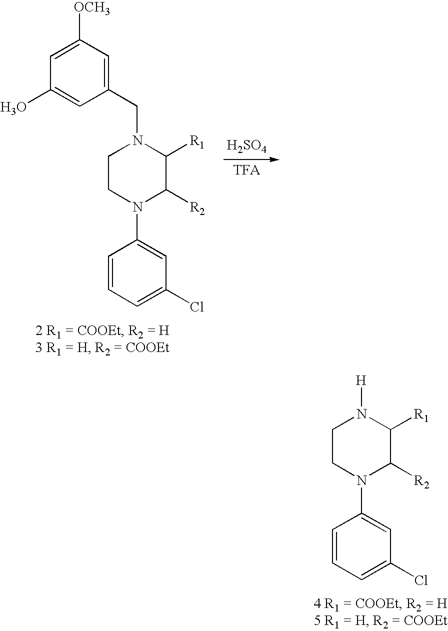

Compound 5. Compound 2 (2.1 g, 5 mmol) and methoxybenzene (1.1 g, 10 mmol) were added to a 5% solution of H₂SO₄ in CF₃COOH (12 mL). After the reaction was heated at 60° C. for 40 h, water (5 mL), 1 N NaOH (10 mL, saturated NaHCO₃ (10 mL), and CH₂Cl₂ (150 mL) were added. The organic layer was separated and dried (Na₂SO₄) and the solvent was removed to give compound 5 (760 mg, 60%). Compound 5 was confirmed by ¹H-NMR and LC-MS.

Compound 4. Compound 4 was prepared from compound 2 in 75% yield following the same procedure as that for compound 5.

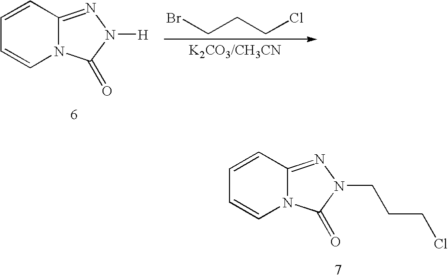

2-(3-chloropropyl)-1,2,4-triazo[4,3-a]pyridin-3(2H)-one(7). A mixture of 1,2,4-triazo[4,3-a]pyridin-3(2H)-one (6), 1.35 g, 10 mmol), 1-bromo-3-chloropropane (4.13 g, 26 mmol) and potassium carbonate (2.07 g, 15 mmol) in MeCN ( 15 mL) was refluxed for 8 h. After removal of the insoluble material by filtration, the filtrate was concentrated and the residue was extracted with CHCl₃ (150 mL). After evaporation of the solvent, the residual material was purified by column chromatography (EtOAc/Heptane, 1:2) to give 2-(3-chloropropyl)-1,2,4-triazo[4,3-a]pyridin-3(2H)-one, compound 7 (1.47 g, 70%). Compound 7 was confirmed by ¹H-NMR and LC-MS.

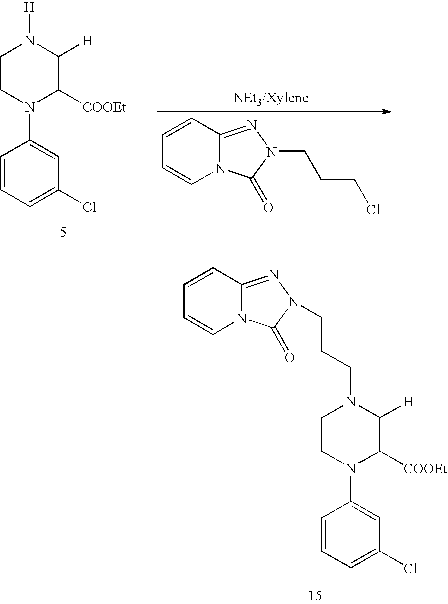

Compound 15. Compound 6, 1.45 g, 6.9 mmol) and 1-(3-chlorophenyl)-2-carbethoxypiperazine (5) (1.75 g, 6.9 mmol) and triethylamine (2 mL, 14.4 mmol) were taken up in xylene (20 mL) and refluxed for 12 h. After cooling to room temperature, the solution was washed with water and evaporated under reduced pressure. The residue was dissolved in EtOAc (100 mL), washed with brine and dried (Na₂SO₄), and was purified by column chromatography (EtOAc/Heptane, 2:3) to give the compound 15 (1.65 g, 65%). Compound 15 was confirmed by ¹H-NMR and LC-MS.

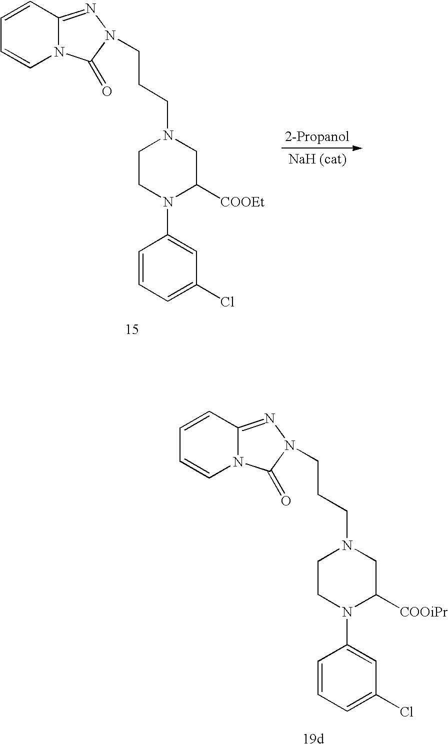

Compound 19d. Sodium hydride (60% dispersion in mineral oil, about 60 mg) was added to a stirred solution of 15 (2.2 g, 4.95 mmol) in 2-propanol (15 mL). After 12 h, the solvent was removed under vacuum. The residue was then dissolved in EtOAc (100 mL), washed with brine, dried (Na2SO4), and purified by column chromatography (EtOAc/Heptane, 1:2) to give the compound 19d (1.58 g, 70%/o). Compound 19d was confirmed by ¹H-NMR and LC-MS.

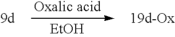

Compound 19d-Oxalate. A solution of oxalic acid (150 mg, 1.68 mmol) in ethanol (1 mL) was added to a stirred solution of compound 19d (770 mg, 1.68 mmol) in ethanol (1.25 mL) in one aliquot. The mixture became solid at the end of the addition and ethyl acetate (2 mL) was added to facilitate stirring. After 1 h of stirring, the solid was collected by suction and washed with ethyl acetate (5 mL). After drying, the oxalate salt 19d-Ox was obtained as white powder (730 mg, 85%). ¹H-NMR and elemental analyses were consistent with the structure.

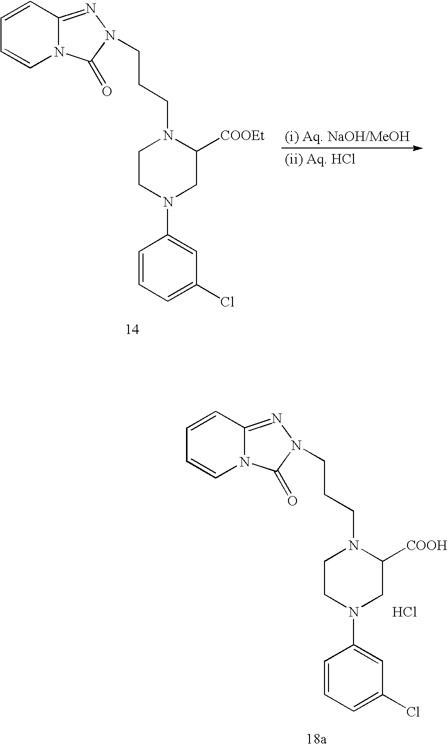

Compound 18a-HCl. Compound 14 (1.2 g, 2.7 mmol) was dissolved in MeOH (20 mL) and an aqueous solution of NaOH (2N) was added. The reaction was refluxed for 2 h and was cooled to room temperature. The solvents were removed and the residue was purified by using preparative HPLC to give the sodium salt of 18a. The sodium salt was dissolved in MeOH (10 mL) and aqueous HCl ( 3 mL, 1 N) was added and stirred for 45 minutes. The solution was concentrated to give the compound 18a-HCl and was confirmed by ¹H-NMR, LC-MS and elemental analysis.

### Trazodone with gem-dimethyl bridge

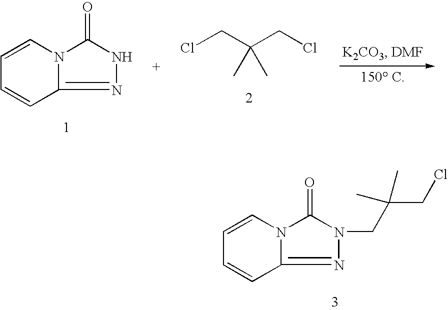

Compound 3. A mixture of 1,2,4-triazolo(4,3-a)pyridin-3(2H)-one (11.2 g, 82.88 mmol) (1), 1,3-dichloro-2,2-dimethyl propane (2), and K₂CO₃ (23.0 g, 21.71 mmol.) in DMF (100 mL) was stirred at 150° C. for 48 hours. Product 3 (5.4 g, 27% yield) was isolated by silica gel column purification and confirmed by ¹H-NMR, LC-MS.

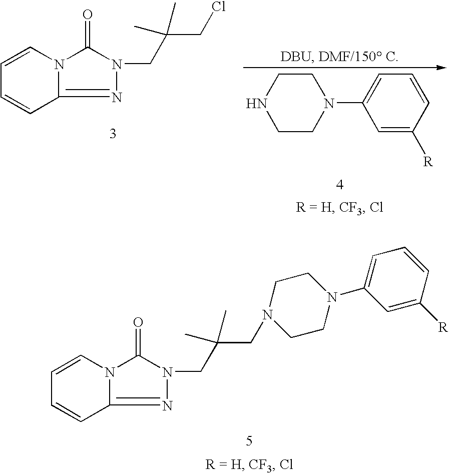

Compound 5. Compound 3 (1.0 equivalent) was dissolved in DMF (25 mL). The amine (4) (1.5 equivalent) and 1,8-diazabicyclo[5.4.0]undec-7-ene (DBU, 1.5 equivalent) were added to the solution, and the solution was heated to 150° C. for 48 h. Product 5, (12% to 20% yield) was isolated after silica gel column chromatography and confirmed by ¹H-NMR, LC-MS.

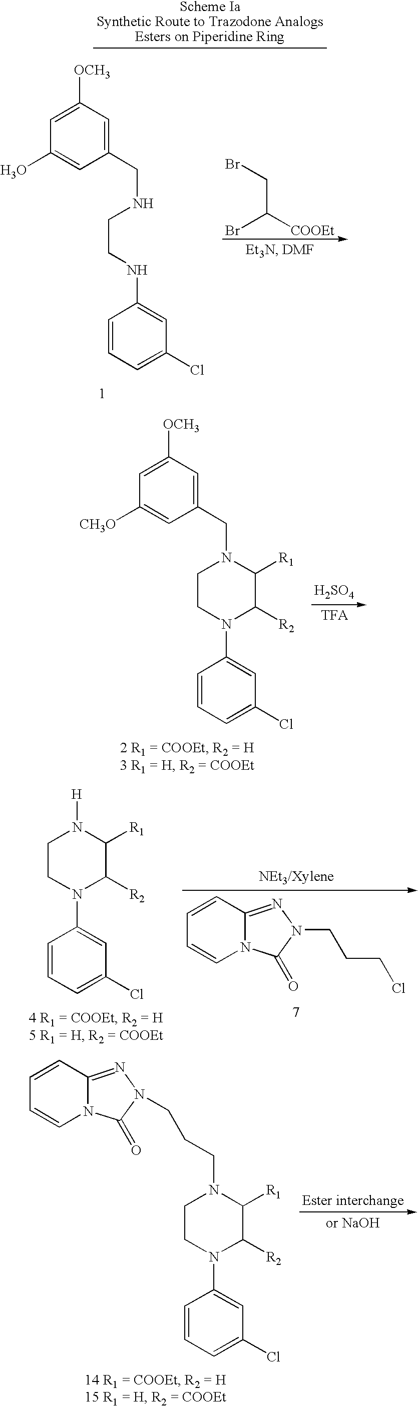

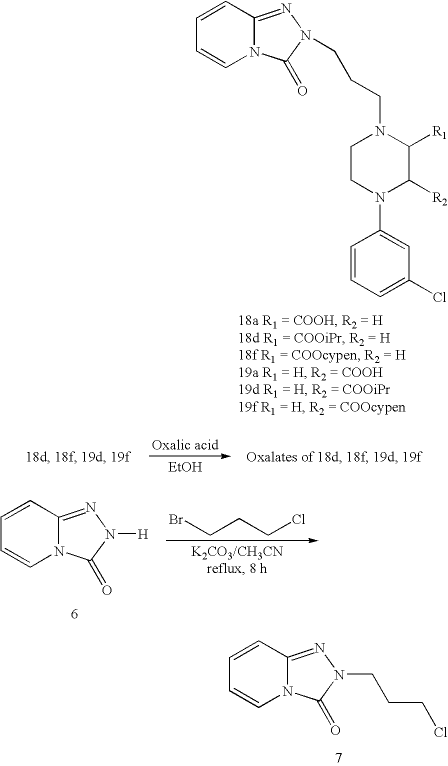

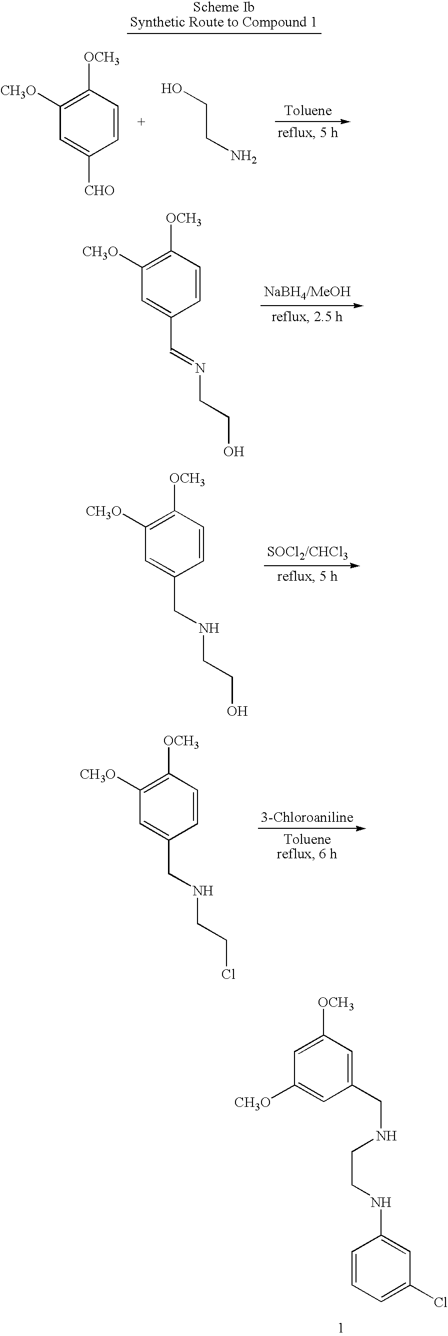

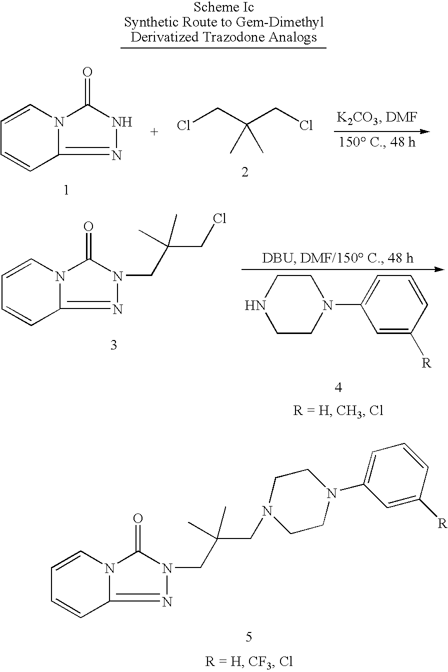

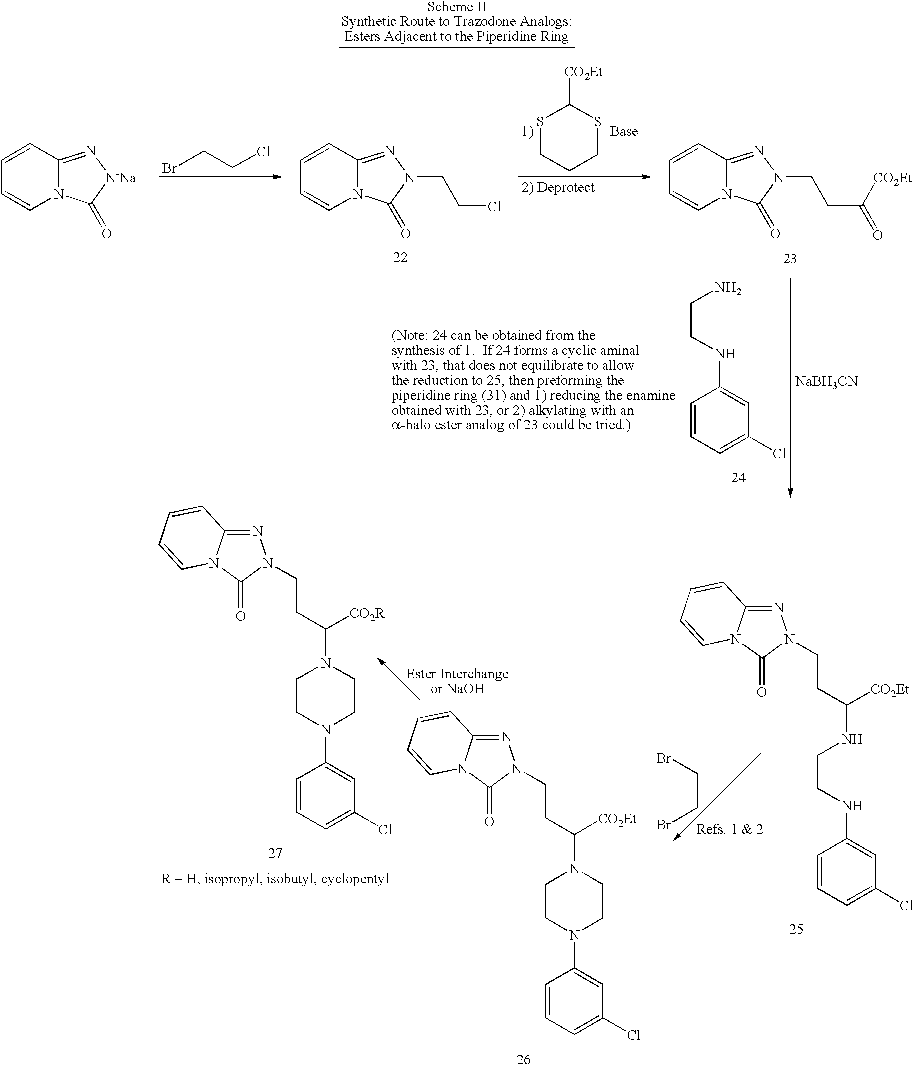

References: 1) G. B. Phillips et al, J. Med. Chem., 1992, 35, 743-750. 2) G. Le Bihan et al, J. Med. Chem., 1999, 42, 1587-1603. 3) M. Giannangeli et al, J. Med Chem., 1999, 42,336-345.

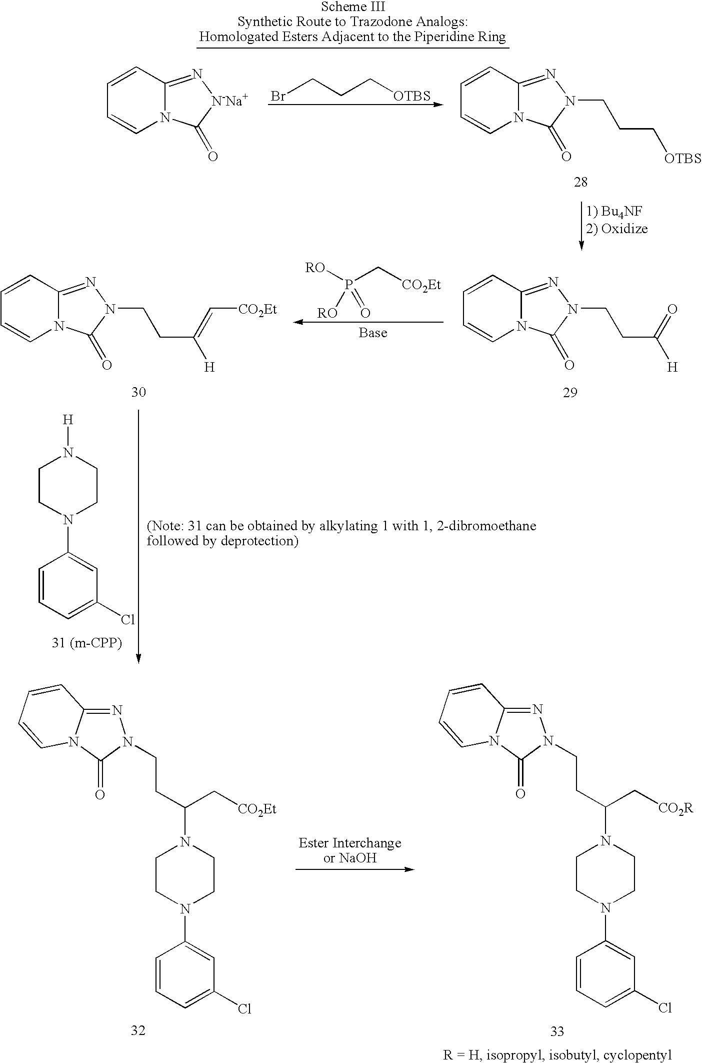

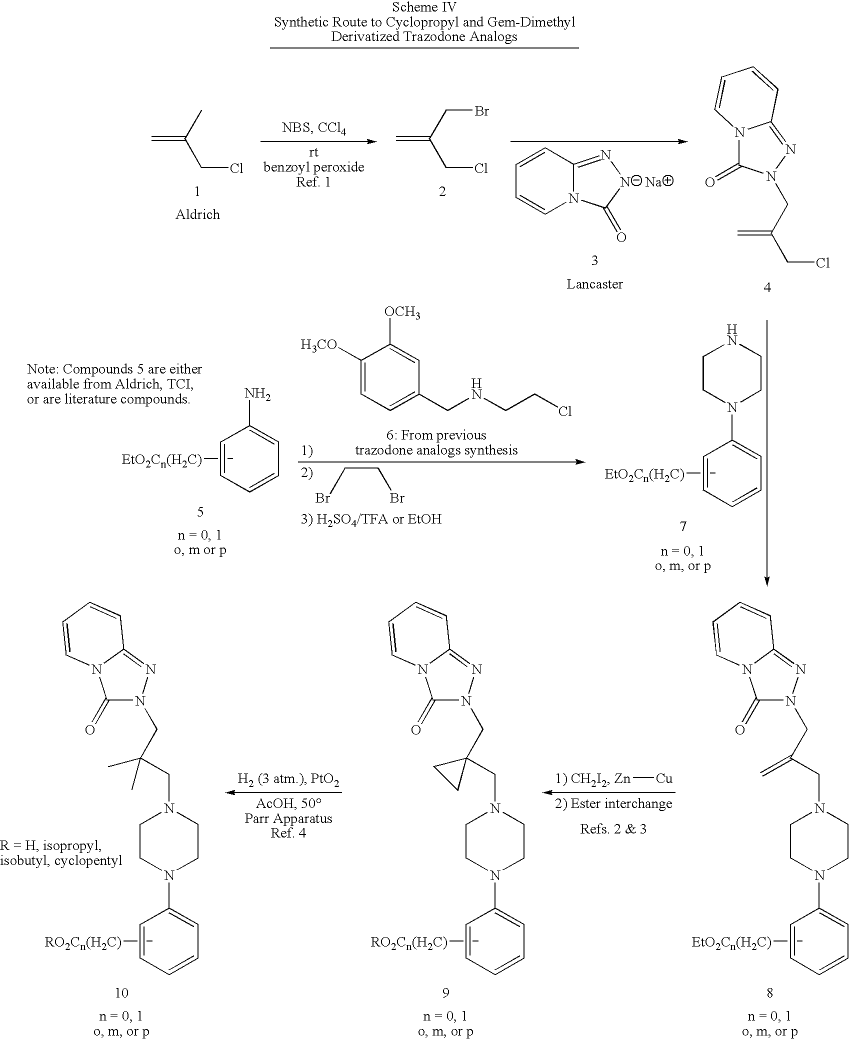

References: 1) Stoller, A. et al, Tetrahedron Lett. (1990), 31(3), 361-4. 2) Motherwell, W. B. et al, Contemp. Org. Synth. (1994), 1(4), 219-241. 3) Simmons, H. E. et al, Org. React. (1973), 20, 1. 4) Woodworth, C. W. et al, Chem. Comm. (1968), 569-570.

### EXAMPLE 2

### Comparison of Trazodone and Trazodone Metabolite Using SCORE-200™

Sleep-wakefulness, locomotor activity and body temperature were monitored in Male Wistar rats treated with Trazodone (10 mg/kg, n=7) and the principal metabolite of Trazodone, m-CPP (3 mg/kg, n=6 and lo mg/kg, n=7). Trazodone was administered at CT-18 (6 hours after lights-off). The Trazodone metabolite m-CPP was administered at CT-5 (5 hours after lights-on). Trazodone disrupted sleep during the first hour but was highly soporific in subsequent hours. Trazodone sleep effects were characterized by increased nonREM sleep time and increased sleep continuity, but without evidence of REM sleep inhibition, rebound insomnia, or disproportional locomotor activity changes. By contrast, the Trazodone metabolite m-CPP significantly interfered with nonREM sleep for 2-3 hours and REM sleep for 7 hours post-treatment. These effects were followed by a rebound hypersomnolence. The temporal course of m-CPP effects on sleep-wakefulness provide working evidence that the initial efficacy and duration of Trazodone action on sleep-wake may be greatly enhanced by inactivating the m-CPP component of Trazodone metabolism through medicinal chemistry modification of the Trazodone molecule.

The general experimental conditions utilized in testing the compounds of the invention for their utility treating sleep disorders are described below.

I. Animals & Surgery.

Adult, male Wistar rats (250 g at time of surgery, Charles River Laboratories) were anesthetized (Nembutal, 62 mg/kg) and surgically prepared with a cranial implant to permit chronic electro-encephalogram (EEG) and electromyogram (EMG) recording. Body temperature and locomotor activity were monitored via a miniature transmitter (Minimitter) surgically placed in the abdomen. The cranial implant consisted of stainless steel screws (two frontal [±3.2 AP from bregma, ±2.0 ML] and two occipital [−6.9 AP, ±5.5 ML]) for EEG recording. Two Teflon-coated stainless steel wires were positioned under the nuchal trapezoid muscles for EMG recording. All leads were soldered to a miniature connector prior to surgery, and gas sterilized in ethylene oxide. The implant assembly was affixed to the skull with dental acrylic. A minimum of three weeks was allowed for surgical recovery.

II. Recording Environment.

Each rat was permanently housed in its own individual recording cage located within separate, ventilated compartments of custom-designed stainless steel cabinets. Each Nalgene microisolator cage was enhanced with a filter-top riser and low-torque swivel-commutator. Food and water were available ad libitum. A 24-hr light-dark cycle (12 hours light, 12 hours dark) was maintained throughout the study using 4-watt fluorescent bulbs 5 cm from the cage. Animals were undisturbed for at least 36 hours before and after treatments.

III. Automated Physiological Monitoring.

Sleep and wakefulness were determined using “SCORE-2000™”—an internet-based sleep-wake and physiological monitoring system. The system monitors amplified EEG (bandpass 1-30 Hz; digitization rate 400 Hz), integrated EMG (bandpass 10-100 Hz), body temperature and non-specific locomotor activity (LMA) via telemetry, and drinking activity, continuously and simultaneously. Arousal states were classified on-line as NREM sleep, REM sleep, wake, or theta-dominated wake every 10 seconds using EEG feature extraction and pattern-matching algorithms. The classification algorithm uses individually-taught EEG-arousal-state templates, plus EMG criteria to differentiate REM sleep from theta-dominated wakefulness, plus behavior-dependent contextual rules (e.g., if the animal was drinking, it was awake). Drinking and locomotor activity (LMA) were recorded as discrete events every 10 seconds, while body temperature was recorded each minute. Locomotor activity was detected by a telemetry receiver (Minimitter, Sunriver, Oregon) beneath the cage. Telemetry measures (LMA and body temperature) were not part of the scoring algorithm; thus, sleep-scoring and telemetry data were independent measures.

IV. Treatments and Study Design.

A. Timing of treatment Compounds were administered at CT-18, the peak of the activity-dominated period, in order to ensure (i) prior wakefulness was sufficient to interact positively with hypnotic-drug effects, and (ii) sufficient time was allowed to view the time course of the treatment effect before lights-on (6 hours post-treatment). The Trazodone metabolite m-CPP was administered at CT-5, the middle of the rodent rest-phase of the daily sleep-wake rhythm, in order to ensure (i) maximum assay sensitivity to the wake-promoting effects of the compound, and (ii) maximum assay sensitivity to compound effects on REM sleep.

B. Vehicle and route of administration. Compounds were suspended in sterile 0.25% methylcellulose (1 ml/kg). Treatments were administered as an intraperitoneal bolus.

C. Study design and controls. A parallel group study design was employed. Vehicle controls were drawn from a large pool (N>200): a subset of the pooled vehicle controls was selected, based on computerized matching with the 24-hour pre-treatment baseline of the active treatment group.

D. Drugs tested. Trazodone and the Trazodone metabolite m-CPP were tested for this proof of principle study. Trazodone was administered at 10 mg/kg. The Trazodone metabolite m-CPP was administered at 3 mg/kg and 10 mg/kg.

V. Results

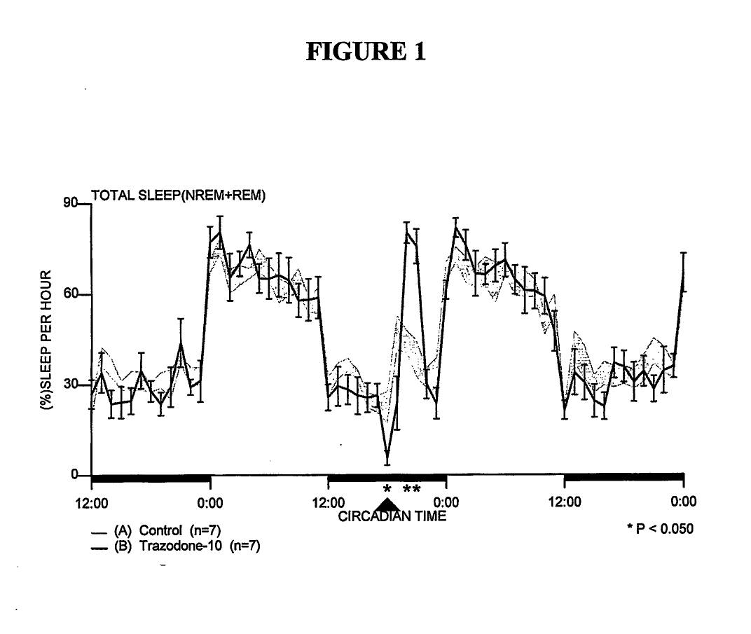

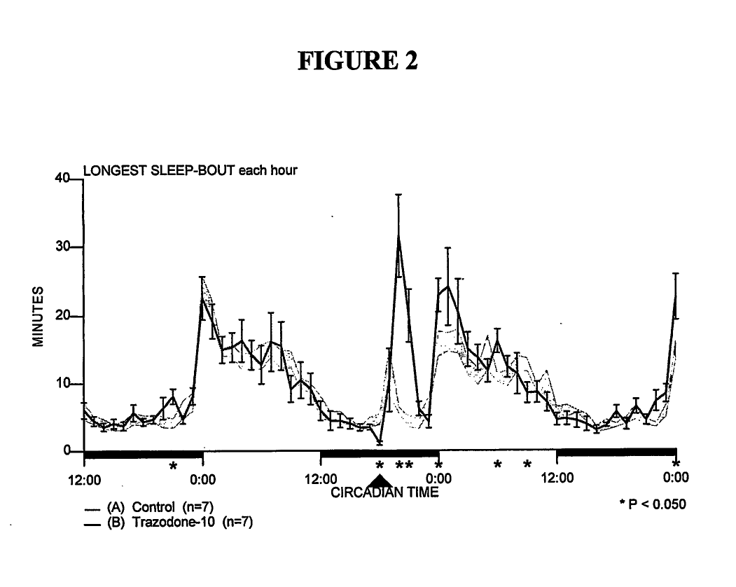

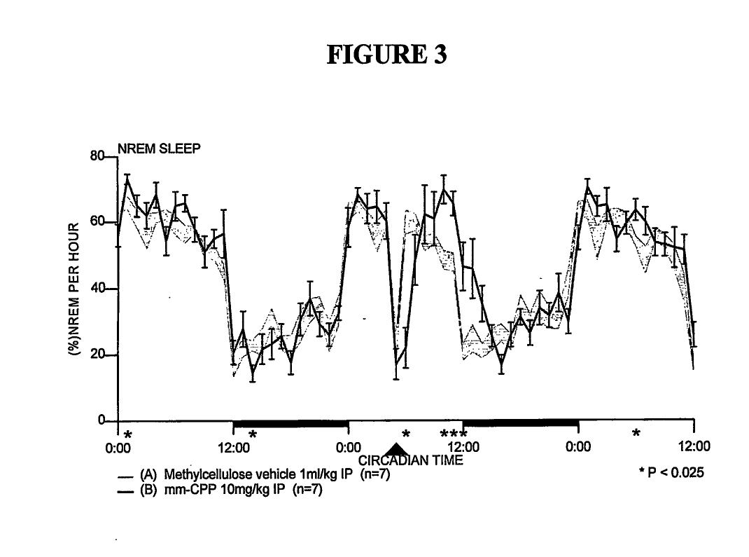

Trazodone (10 mg/kg IP, n=7) interfered with sleep during the initial 2 hours post-treatment but markedly and significantly increased nonREM sleep time (FIG. 1) and sleep bout duration for 2-3 hours thereafter (FIG. 2), and increased sleep consolidation in the initial 2-3 hours of the subsequent subjective day (lights-on phase of the LD 12:12 light-dark cycle). Trazodone produced no evidence of rebound insomnia, disproportional motor inhibition, or adverse thermoregulatory events post-treatment. The sleep consolidating effects of Trazodone were especially noteworthy, as they were more robust in magnitude when compared to comparable treatment with contemporary benzodiazepine sedative hypnotics such as zolpidem.

[image]

[image]

[image]

The Trazodone metabolite m-CPP (3 mg/kg IP, n=6, and 10 mg/kg IP, n=7) strongly and dose-dependently interfered with sleep 2-3 hours post-treatment. Interference with sleep was characterized by a dose-dependent reduction in nonREM sleep time lasting 2-3 hours post-treatment, and a marked dose-dependent reduction in REM sleep lasting up to7 hours post-treatment at the higher dose. Sleep interference caused by m-CPP was followed by rebound hypersomnolence reflected in both nonREM sleep and sleep bout-length measures. The timecourse of sleep interference (increased waking) caused by m-CPP correlated very strongly with the initial interference of sleep following Trazodone treatment (noted above). In addition, the rebound hypersomnolence caused by m-CPP correlated very strongly with the timecourse of carryover effects following Trazodone administration (noted above). Taken together, it is likely that the delayed onset of Trazodone-induced sleep is caused in part or completely by the sleep-interference characteristics of the Trazodone metabolite m-CPP. It is further likely that the soporific carryover effects of Trazodone are caused in part or completely by the rebound hypersomnolence induced by the Trazodone metabolite m-CPP.

V. Conclusions

Trazodone has considerable potential as a sedative hypnotic if the undesirable effects of the Trazodone metabolite m-CPP (sleep interference, rebound hypersomnolence, REM sleep inhibition and sympathomimetic effects) could be inactivated through medicinal chemistry modification of the Trazodone molecule. On the basis of the data from this study, it is anticipated that the efficacy of Trazodone will be increased, and drug carry-over will be decreased, through inactivation of the Trazodone metabolite m-CPP.

### EXAMPLE 3

### Comparison of Trazodone and Trazodone Analog Using SCORE-2000™

Sleep-wakefulness, locomotor activity and body temperature were monitored in Male Wistar rats treated with Trazodone (30 mg/kg, n=9) and HY-2725 (I9) (30 mg/kg, n=8). The general experimental conditions utilized in testing the compounds of the invention for their utility treating sleep disorders are described in Example 2.

Results

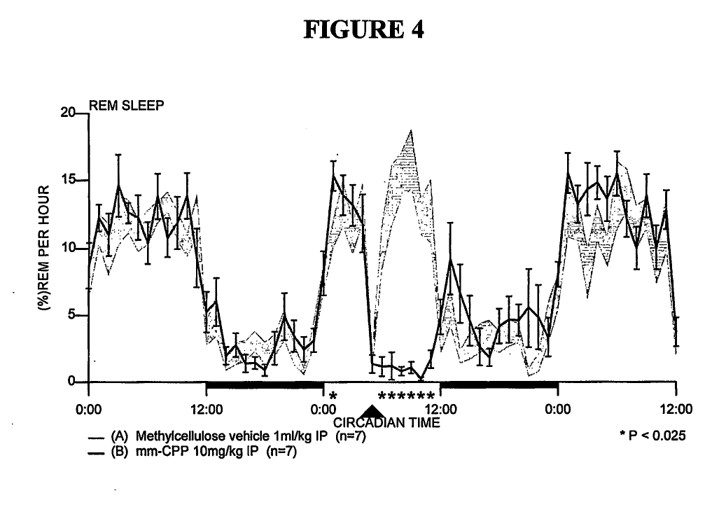

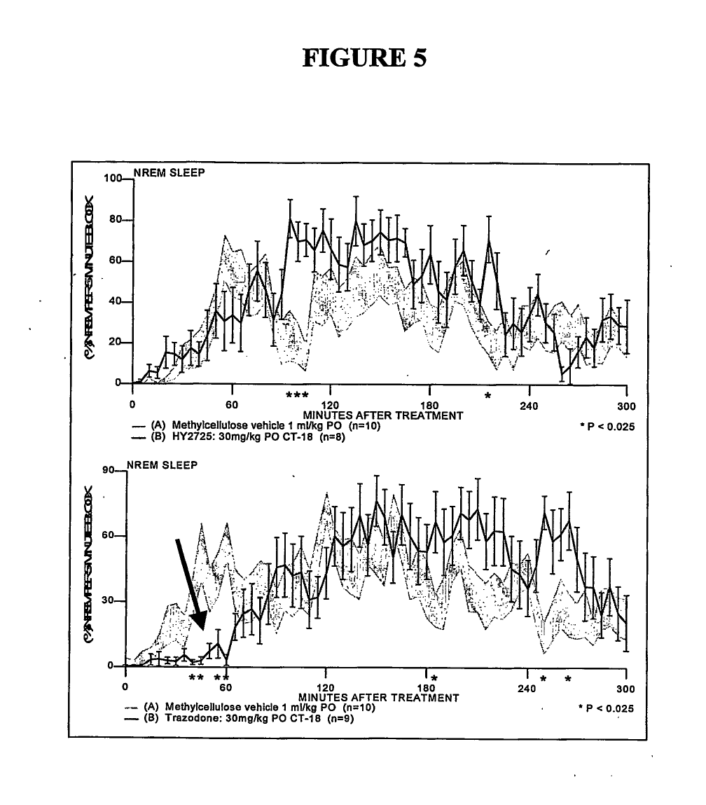

Trazodone initially interferes with sleep (FIG. 5: arrow; lower plot) whereas HY-2725 has a more rapid soporific onset of action and does not interfere with sleep (FIG. 2: upper plot). The initial interference in sleep after trazodone treatment is believed to be caused by the formation of the Trazodone metabolite m-CPP. HY-2725 is designed to reduce or eliminate the formation of this metabolite.

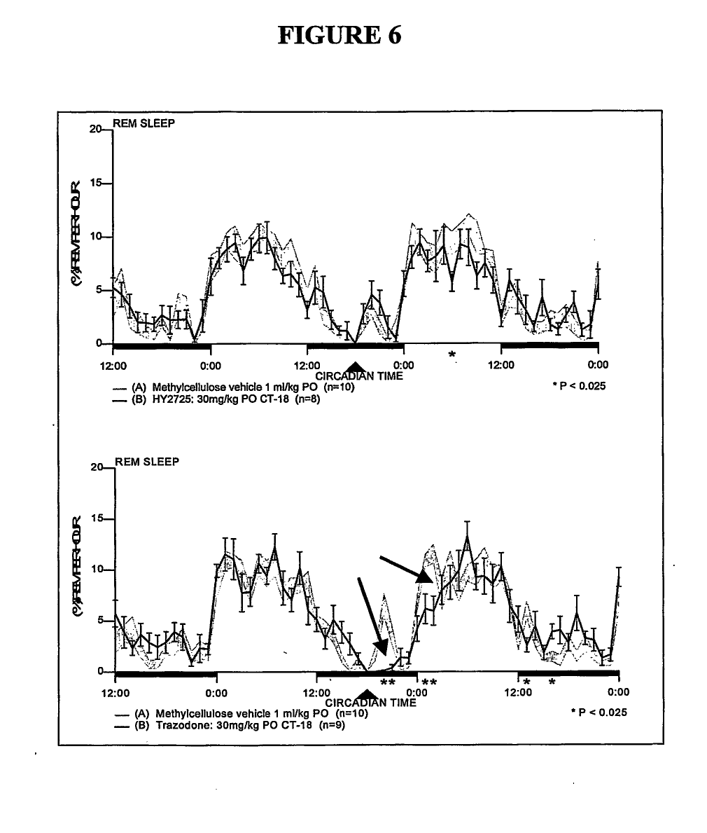

FIG. 6 demonstrates that Trazodone treatment (triangle) inhibits REM sleep (FIG. 6: arrows, lower plot), whereas HY-2725 does not inhibit REM sleep.

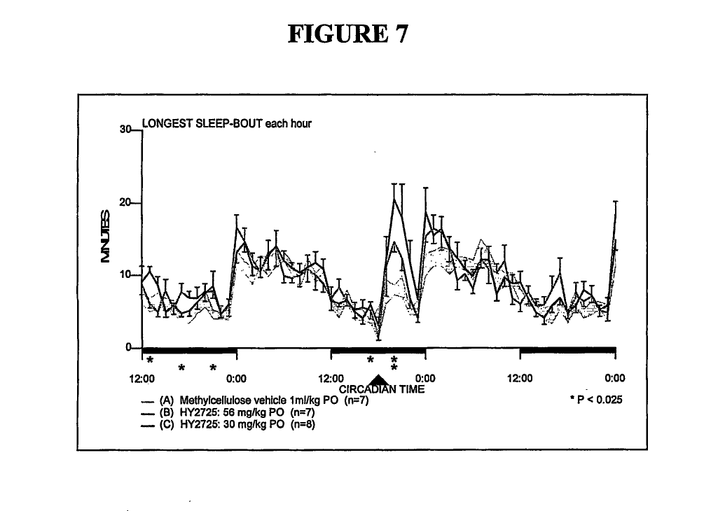

In addition, HY-2725, a cyclopentyl ester analog, potently and dose-dependently increases sleep consolidation after treatment (FIG. 7: triangle).

[image]

[image]

[image]

Several interesting SCORE components determined for the parent Trazodone compound and Compound 19f are shown below. Compound 19f shows not initial sleep interference and no REM sleep inhibition, whereas Trazodone shows significant initial sleep interference and significant REM sleep inhibition. In addition the duration of action of 19f is significantly decreased as compared with Trazodone.

Summary of Findings using the SCORE-2000™ Sleep-Wake Assay:

| | | Compound |
|---|---|---|
| | TRAZODONE | 19f |
| Initial Sleep Interference | ++++ | None |
| REM Sleep Inhibition | ++++ | None |
| Increase Sleep Consolidation | +++ | ++++ |
| Increase Sleep Time | +++ | +++ |
| Rebound Insomnia | None | None |
| Disproportional Motor Inhibition | None | None |
| Body Temp (CV) Adverse Event | Yes | No |
| Duration of Action | 7-9 h | 5-6 h |

Conclusions

As discussed in Example 2, Trazodone has considerable potential as a sedative hypnotic if the undesirable effects of the Trazodone metabolite m-CPP (sleep interference, rebound hypersomnolence, REM sleep inhibition and sympathomimetic effects) could be inactivated through medicinal chemistry modification of the Trazodone molecule. On the basis of the data from this study, including the experimental results obtained for Compound 19f, it is anticipated that the efficacy of Trazodone will be increased, and drug carry-over will be decreased, through inactivation of the Trazodone metabolite m-CPP.

### EXAMPLE 4

### Determination of Activity of Carboxylic Acid Derivatized Trazodone Compound

Sleep-wakefulness, locomotor activity and body temperature were monitored in Male Wistar rats treated with HY-2724 (a) (30 mg/kg, n=7). The general experimental conditions utilized in testing the compounds of the invention for their utility treating sleep disorders are described in Example 2.

Results

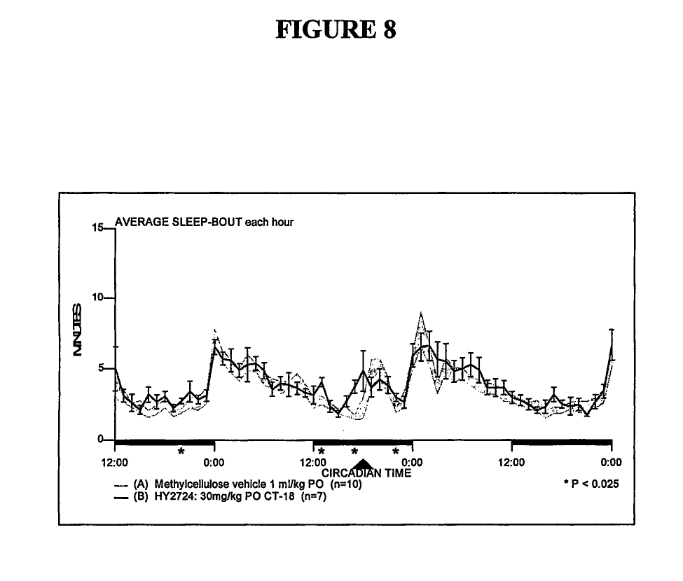

FIG. 8 shows that the acid (L9a) form of HY-2725 (9f; cyclopentyl ester) is ineffective in increasing sleep and sleep consolidation. HY-2724 was inactive on sleep-wakefulness in all measured variables.

[image]

Conclusions

On the basis of the data from this study, it would appear that the corresponding acid becomes inactive once metabolized from the ester to the acid form, e.g., by esterases. This “deactivation” of the active compound should provide an ability to sufficiently control (modify) the half-life of the ester derivatized compounds.

### EXAMPLE 5

### 5-HT_{2A} Binding Study

Binding assays were performed on Trazodone, HY-2725 (19f), HY-2650 (19d) and HY-2724 (19a), described above, using both rat and human 5-HY_{2A} receptor. The results are shown in Table 3.

The binding studies against the 5-HT_{2A} receptor, indicate binding affinity, and therefore the results of the binding assays are an indication of the activity of the compound.

Table 3 shows rat and human 5-HT_{2A} receptor binding for the above-identified compounds. Soporific efficacy and sleep consolidation paralleled binding affinity at 5-HT_{2A} for HY-2725, HY-2650 and HY-2724. Although HY-2725 binding affinity is shown as less than that of Trazodone, however, the HY2725 compound used was a racemic mixture of two isomers. Thus, the effective binding affinity of HY-2725 may be equal or nearly equal to that of Trazodone. It is hypothesized that HY-2725 soporific efficacy is superior to Trazodone because HY-2725 does not produce the metabolite m-CPP.

TABLE 3

| | | 5HT-2a (Ki nM) | |
|---|---|---|---|
| Compound | Side-Chain | Rat | Human |
| Trazodone | none | 8.11 | 286 |
| HY-2725 | cyclopentyl | 18.7 | 757 |
| HY-2650 | isapropyl | 50.3 | 2,103 |
| HY-2724 | acid | 989 | >10,000 |

### INCORPORATED BY REFERENCE

The entire contents of all patents, published patent applications and other references cited herein are hereby expressly incorporated herein in their entireties by reference.

Equivalents

Those skilled in the art will recognize, or be able to ascertain, using no more than routine experimentation, many equivalents to specific embodiments of the invention described specifically herein. Such equivalents are intended to be encompassed in the scope of the following claims.

1.-128. (canceled)

129. A method of treating a sleep disorder, comprising administering to a subject an effective amount of a trazodone compound, such that the sleep disorder is treated, wherein said trazodone compound is represented by the formula: 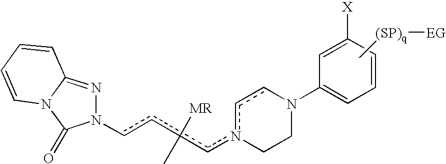
wherein MR is a metabolite reducing moiety that reduces the formation of wake-promoting metabolites, MR is attached to a carbon along the dotted line shown, EG is an ester group that modifies the half-life of the trazodone compound, SP is a spacer molecule, q is 0 or 1, X is H or Cl.

130. The method of claim 129, wherein said spacer molecule is (CH₂)ₘ, where m is an integer selected from 1 to 20.

131. The method of claim 129, wherein said trazodone compound containing MR is more effective as a therapeutic agent for treating a sleep disorder than the corresponding compound without the MR.

132. The method of claim 129, wherein said trazodone compound containing said EG is more effective as a therapeutic agent for treating a sleep disorder than the corresponding compound without the EG.

133. The method of claim 129, wherein said trazodone compound containing EG is more effective as a therapeutic agent for treating a sleep disorder than the corresponding acid of said EG.

134. The method of claim 129, wherein said trazodone compound containing the corresponding acid of EG is not a therapeutically effective agent for treating a sleep disorder.

135. The method of claim 129, wherein said wake promoting metabolite is m-CPP.

136. The method of claim 129, wherein the trazodone compound induces a discrete sleep or hypnotic state by penetration into the Central Nervous System (CNS).

137. The method of claim 129, wherein the sleep disorder is selected from the group consisting of insomnia, hypersomnia, narcolepsy, sleep apnea syndromes, parasomnia, restless leg syndrome, and circadian rhythm abnormality.

138. The method of claim 137, wherein the sleep disorder is insomnia.

139. The method of claim 137, wherein the sleep disorder is hypersomnia.

140. The method of claim 137, wherein the sleep disorder is narcolepsy.

141. The method of claim 137, wherein the sleep disorder is sleep apnea syndrome.

142. The method of claim 137, wherein the sleep disorder is parasomnia.

143. The method of claim 137, wherein the sleep disorder is restless leg syndrome.

144. The method of claim 137, wherein the sleep disorder is circadian rhythm abnormality.

145. The method of claim 144, wherein the circadian rhythm abnormality is selected from the group consisting of jet lag, shift-work disorders, and delayed or advanced sleep phase syndrome.

146. The method of claim 129, wherein the trazodone compound is administered orally.

147. The method of claim 129, further comprising administering the trazodone compound in a pharmaceutically acceptable vehicle.

148. The method of claim 129, wherein MR is one or more moieties that are attached at one or more positions along the dotted line.

149. The method of claim 148, wherein MR is a single moiety that is attached at multiple positions.

150. The method of claim 148, wherein MR is more than one moiety attached at multiple positions.

151. The method of claim 129, wherein MR is an alkyl group.

152. The method of claim 129, wherein MR is selected from the MRs represented in the compounds listed in Table 2.

153. The method of claim 152, wherein MR is selected from a methyl, a geminal dimethyl, a cyclopropyl, a COOH, a COO-ethyl, a COO-isopropyl, a COO-cyclopentyl, a COO-pentyl, a cycloheptyl, and a benzyl group.

154. The method of claim 149, wherein MR is cyclopropyl.

155. A method of treating a sleep disorder, comprising administering to a subject an effective amount of a trazodone compound, such that the sleep disorder is treated, wherein said trazodone compound is represented by the formula: 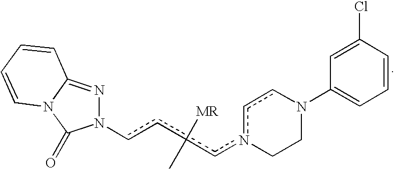
wherein MR is selected from a geminal dimethyl, a cyclopropyl, a COOH, a COO-ethyl, a COO-isopropyl, a COO-cyclopentyl, a COO-pentyl, a cycloheptyl, and a benzyl group.

156. A method of treating a sleep disorder, comprising administering to a subject an effective amount of a trazodone compound, such that the sleep disorder is treated, wherein said trazodone compound is selected from the group consisting of: 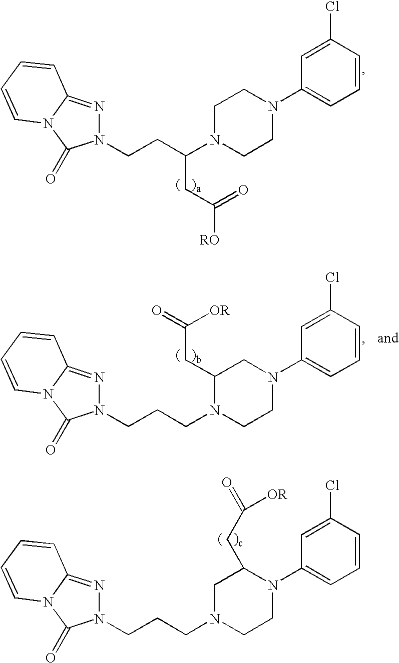
wherein a, b, and c are, independently, selected from 0, 1, 2, 3, 4, and S. and R is any group which imparts properties to the trazodone compound to promote reduction of formation of wake-promoting metabolites, and modification to the half-life of the compound.

157. The method of claim 156, wherein a is 0 or 1.

158. The method of claim 156, wherein b is 0 or 1.

159. The method of claim 156, wherein c is 0 or 1.

160. The method of claim 156, wherein R is selected from the group consisting of hydrocarbons and perfluorocarbons.

161. The method of claim 160, wherein the hydrocarbons are selected from the group consisting of linear; branched; cyclic; aromatic; and a combination of saturated or unsaturated aliphatic and aromatic; wherein further the hydrocarbons are optionally substituted with O, N, S, or halogen and may additionally include one or more centers of chirality.

162. The method of claim 160, wherein the hydrocarbons contain from 1 to 20 carbons.

163. The method of claim 156, wherein R is selected from the group consisting of a methyl, an ethyl, an n-propyl, an isopropyl, a t-butyl, an isobutyl, a cyclopentyl, a cyclohexyl, a cycloheptyl, and a benzyl group.

164. The method of claim 163, wherein R is a cyclohexyl group.

165. The method of claim 163, wherein R is a cyclopentyl group.

166. The method of claim 163, wherein R is a cycloheptyl group.

167. The method of claim 163, wherein R is an isobutyl group.

168. The method of claim 163, wherein R is an ethyl group.

169. The method of claim 163, wherein R is a methyl group.

170. The method of claim 163, wherein R is an n-propyl group.

171. The method of claim 163, wherein R is an isopropyl group.

172. The method of claim 163, wherein R is a t-butyl group.

173. The method of claim 163, wherein R is a benzyl group.

174. The method of claim 163, wherein R is a bulky alcohol.

175. The method of claim 174, wherein the bulky alcohol is selected from the alcohols listed in Table 1.

176. A method of treating a sleep disorder, comprising administering to a subject an effective amount of a trazodone compound, such that the sleep disorder is treated, wherein said trazodone compound is represented by the formula: 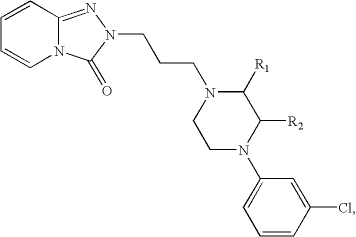
wherein R₁ and R₂ are, independently, selected from H, COO-isopropyl, and COO-cyclopentyl, provided that at least one of R₁ and R₂ is not H.

177. The method of claim 176, wherein one of R₁ and R₂ is H.

178. A compound of the formula: 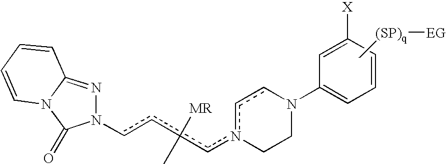
wherein MR is a metabolite reducing moiety that reduces the formation of wake-promoting metabolites, MR is attached to a carbon along the dotted line shown, EG is an ester group that modifies the half-life of the trazodone compound, SP is a spacer molecule, q is 0 or 1, X is H or Cl.

179. The compound of claim 178, wherein said wake promoting metabolite is m-CPP.

180. The compound of claim 178, wherein said spacer molecule is (CH₂)ₘ, where m is an integer selected from 1 to 20.

181. The compound of claim 178, wherein MR is one or more moieties attached at one or more positions along the dotted line.

182. The compound of claim 181 wherein MR is a single moiety that is attached at multiple positions.

183. The compound of claim 181, wherein MR is more than one moiety attached at multiple positions.

184. The compound of claim 178, wherein MR is an alkyl group.

185. The compound of claim 178, wherein MR is selected from the MRs represented in the compounds listed in Table 2.

186. The compound of claim 185, wherein MR is selected from methyl, geminal dimethyl, cyclopropyl, COOH, COO-ethyl, COO-isopropyl, COO-cyclopentyl, COO-pentyl a cycloheptyl, and benzyl.

187. The compound of claim 181, wherein MR is cyclopropyl.

188. A compound represented by the formula: 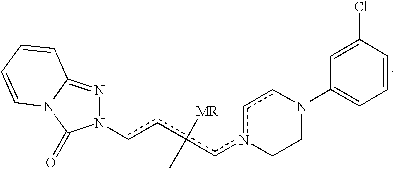
wherein MR is selected from a geminal dimethyl, a cyclopropyl, a COOH, a COO-ethyl, a COO-isopropyl, a COO-cyclopentyl, a COO-pentyl, a cycloheptyl, and a benzyl group.

189. A compound selected from: 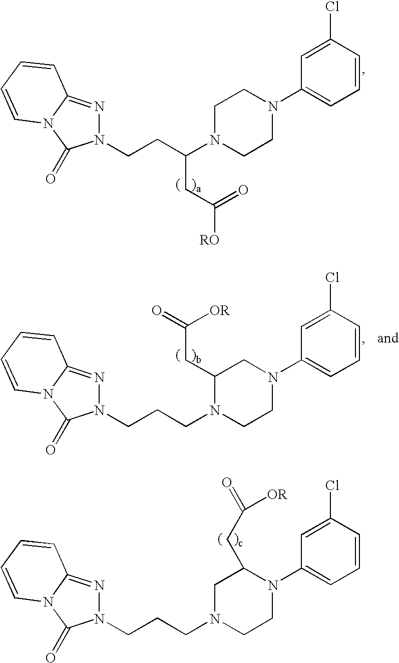
wherein a, b, and c, are, independently selected from 0, 1, 2, 3, 4, and 5, and R is any group which imparts properties to the trazodone compound to promote reduction of formation of wake-promoting metabolites, and modification to the half-life of the compound.

190. The compound of claim 189, wherein a is 0 or 1.

191. The compound of claim 189, wherein b is 0 or 1.

192. The compound of claim 189, wherein c is 0 or 1.

193. The compound of claim 189, wherein R is selected from the group consisting of hydrocarbons and perfluorocarbons.

194. The compound of claim 193, wherein the hydrocarbons are selected from the group consisting of linear; branched; cyclic; aromatic; and a combination of saturated or unsaturated aliphatic and aromatic; wherein further the hydrocarbons are optionally substituted with O, N, S, or halogen and may additionally include one or more centers of chirality.

195. The compound of claim 193, wherein the hydrocarbons contain from 1 to 20 carbons.

196. The compound of claim 189, wherein R is selected from the group consisting of a methyl, an ethyl, an n-propyl, an isopropyl, an n-butyl, a t-butyl, a cyclopentyl, a cyclohexyl, a cycloheptyl, and a benzyl group.

197. The compound of claim 196 wherein R is a cyclohexyl group.

198. The compound of claim 196, wherein R is a cyclopentyl group.

199. The compound of claim 196, wherein R is a cycloheptyl group.

200. The compound of claim 196, wherein R is an isobutyl group.

201. The compound of claim 196, wherein R is an ethyl group.

202. The compound of claim 196, wherein R is a methyl group.

203. The compound of claim 196, wherein R is an n-propyl group.

204. The compound of claim 196, wherein R is an isopropyl group.

205. The compound of claim 196, wherein R is a t-butyl group.

206. The compound of claim 196, wherein R is a benzyl group.

207. The compound of claim 178, wherein said compound is formulated to provide controlled in vivo absorption of the compound over a discrete period of time.

208. A compound having the formula: 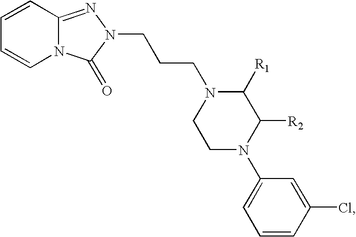
wherein R₁ and R₂ are, independently, selected from H, COO-isopropyl, and COO-cyclopentyl, provided that at least one of R₁ and R₂ is not H.

209. The compound of claim 208, wherein one of R₁ and R₂ is H.
